(19) United States
(12) Patent Application Publication
(10) Pub. No.: US 2006/0078919 A1
(43) Pub. Date: Apr. 13, 2006

(54) Diagnostic test kit and methods of diagnosis and treatment of helicobacter SPP. associated infections

(75) Inventors: James R. Musick, Conifer, CO (US); Harry C. Gurney, Conifer, CO (US); Janice Gurney, Conifer, CO (US)

Correspondence Address: PATTON BOGGS, 1660 LINCOLN ST, SUITE 2050, DENVER, CO, 80264, US

(73) Assignee: Harry C. Gurney Janice Gurney, Conifer, CO (US)

(21) Appl. No.: 11/222,064

(22) Filed: Sep. 8, 2005

Related U.S. Application Data
(60) Provisional application No. 60/608,346, filed on Sep. 8, 2004.

(51) Int. Cl. C12Q 1/68; C07H 21/04

(52) U.S. Cl. 435/6; 536/25.32

(57) ABSTRACT

The present diagnostic test kit and methods of diagnosis and treatment of Helicobacter spp. associated infections provides accurate diagnosis and treatment for Helicobacter spp. related infections and diseases. The method of diagnosis includes analyzing a tissue specimen from a patient diagnosed with a disease. The tissue specimen can be a tumor cell related to the diagnosed disease of the patient. The tissue specimen is analyzed for the presence of Helicobacter spp. DNA or its components. One such analysis includes fluorescent in-situ hybridization (FISH). Once the presence of Helicobacter spp. DNA and/or its components is detected, the patient may be treated with a host of treatments including antibiotic therapy, molecular signal therapy, vaccination, interference with receptor systems mediating Helicobacter spp. invasion into tissues, and use of small-interfering RNA sequences to specifically block virulence or replication of Helicobacter spp. These treatments are administered for a duration to eradicate the Helicobacter spp. in the tissues of the patient. A test kit is also provided for quickly determining the presence of unspecified Helicobacter spp. DNA in a tissue specimen of a patient.

### CROSS-REFERENCE TO RELATED APPLICATIONS

This application claims the benefit of U.S. Provisional Application No. 60/608,346 filed 8 Sep. 2004 and titled, “Diagnosis and Treatment of Non-Gastric Helicobacter Infections.”

### FIELD OF THE INVENTION

This invention relates to the etiological involvement of the Helicobacter subspecies (spp.) microorganisms of various strains in conditions other than gastrointestinal inflammation and ulcers of the stomach and duodenum. The invention further relates to the use of diagnostic tests and therapeutic monitoring for Helicobacter spp. organisms using molecular testing for specific components of Helicobacter spp. organisms using molecular testing for specific components of the Helicobacter spp. toxins or virulence factors elaborated by these organisms. In addition, the invention further relates to the therapeutic treatment of the non-gastric Helicobacter infections.

### Problem

Initial screening for the presence of a Helicobacter spp. organism presently involves the use of blood tests to detect the presence of antibodies, such as IgG, to common Helicobacter spp. Some exemplary screening tests include, lateral flow qualitative immunoassay tests according to commercially available products such as, QuickVue®, H. pylori gII™; and Quidel®. There are difficulties with such tests that limit their accuracy between cats, dogs, and humans and between strain differences of Helicobacter spp. These difficulties include spurious false negative tests by otherwise symptomatic patients and spurious positive tests where there is a prolonged or lingering presence of reactive antibodies for a significant period of time after the infection in the patient has been eliminated. Such tests are therefore subject to considerable error from both false positives and false negatives.

Another problem affecting accurate diagnoses of Helicobacter spp. of non-gastric infections relates to normal presence of Helicobacter spp. organisms within the gastrointestinal (GI) tract of a patient. Prior art as well as recent studies show that Helicobacter spp. organisms are often present within the GI tract, and more particularly the upper GI tract including the buccal cavity, saliva, and the stomach. An emerging concept is that Helicobacter spp. is a normal floral constituent of the GI tract as is the case with certain species of bacteria such as E. coli.

Further, there exists a problem with treating animals, such as humans, dogs, and cats, with conditions and diseases that are not limited to the GI tract. These conditions and diseases may include: benign and malignant non-gastric tumors (identified as adenocarcinoma of the stomach of dogs and humans), rheumatoid arthritis, macular degeneration, retinitis, glaucoma, “dry eye”, acute and chronic degenerative liver disease, acute and chronic kidney disease (cystic fibrosis and amyloidosis), diabetes mellitus (Type I and II), periodontal disease, acute and chronic gingivitis, cardiomyopathy, gastroesophageal reflux disease (GERD), spinal demyelinization, multiple sclerosis, and various forms of pemphigoid diseases, and other immune-mediated diseases (so called, auto-immune diseases).

Therefore, there is a need for a reliable method for determining the cause of diagnosed non-gastric disease in a patient by a more precise and reliable testing method. In addition, there is a need for a reliable method for detecting the presence of Helicobacter spp. within bodily tissues and fluids located throughout the body to enable the determination of the cause of the diagnosed non-gastric disease in a patient. Moreover, there is a need for therapeutic treatment for many conditions and diseases caused by the presence of Helicobacter spp.

### Solution

The above-described problems are solved and a technical advance is achieved in the art by the present diagnostic test kit and method of diagnosis and treatment of Helicobacter spp. associated infections. The invention relates to the use of diagnostic tests and therapeutic in-situ monitoring for Helicobacter spp. organisms using molecular testing for specific components of Helicobacter spp. and toxins or metabolic virulence factors elaborated by these organisms.

This invention further demonstrates the etiological involvement of the Helicobacter spp. microorganisms in various animal species including, humans, dogs, and cats, in conditions not limited to the GI tract, such as: benign and malignant non-gastric tumors (identified as adenocarcinoma of stomach of dogs and humans), rheumatoid arthritis, macular degeneration, retinitis, glaucoma, “dry eye”, acute and chronic degenerative liver disease, acute and chronic kidney disease (cystic fibrosis and amyloidosis), diabetes mellitus (Type I and II), periodontal disease, acute and chronic gingivitis, cardiomyopathy, gastroesophageal reflux disease (GERD), spinal demyelinization, multiple sclerosis, various forms of pemphigoid diseases, and other immune-mediated diseases (so called, auto-immune diseases).

One aspect of the present invention is based on the discovery that Helicobacter spp. organisms of various strains are associated with diseases other than those traditionally known to be caused by Helicobacter spp. infection, such as duodenal and gastric ulcer, gastritis, and some forms of gastric cancer. From this discovery, therapies were designed to treat diseases safely involving the eradication, blocking, modulation, and/or neutralization of the Helicobacter spp. infection. Such therapeutic interventions were found to be of benefit in the treatment of specific diseases indicating an etiological role of Helicobacter spp. pathogenesis in these conditions. Treatment modalities include specific antibiotic regimens, use of Helicobacter spp. toxins and virulence factors and their derivatives in molecular signaling therapy, therapeutic intervention by interference with host receptors to the Helicobacter spp. organism and/or toxin or virulence factors, vaccines, and small interfering RNA.

### SUMMARY

The invention provides a method for determining the cause of a diagnosed non-gastric disease in a patient, including: obtaining a tissue specimen related to the diagnosed non-gastric disease from the patient; synthesizing a single strand of a double strand DNA Helicobacter specific nucleotidic sequence of the tissue specimen for binding a fluorescent moiety on the single strand of the double strand of DNA of the nucleotidic sequence; denaturing the DNA nucleotidic sequence to separate the double strand of DNA to allow access of the fluorescent moiety to their respective nucleotidic sequence; detecting the tissue specimen for the presence of a Helicobacter spp.; and associating a presence of the Helicobacter spp. with the diagnosed non-gastric disease. Preferably, the fluorescent moiety is selected from the group consisting of FITC, 6FAM, TRITC, Texas red, and rhodamine. Preferably, the binding a fluorescent moiety includes covalently attaching the fluorescent moiety to the 5′ end of the DNA sequence 5′-CAC-ACC-TGA-CTG-ACT-ATC-CCG-3′ of the nucleotidic sequence. Preferably, the binding a fluorescent moiety includes covalently attaching the fluorescent moiety to the 5′ end of the DNA sequence 5′-GCC-GTG-CAG-CAC-CTG-TTT-TCA-3′ of the nucleotidic sequence. Preferably, detecting the tissue specimen includes subjecting the fluorescent moiety of the tissue specimen to a light source having an excitation wavelength to cause the fluorescent moiety emit an emission wavelength.

Preferably, the excitation wavelength is substantially between 280 nm and 650 nm. Preferably, detecting the tissue specimen further includes detecting the emission wavelength to determine the presence of Helicobacter spp. Preferably, the nucleotidic sequences is selected from the group consisting of Helicobacter species-specific sequences, Helicobacter genus-specific sequences, altered sequences that do not bind to the Helicobacter target sequences, and sequences binding to antibiotic resistant strains of Helicobacter spp. Preferably, the invention further includes detecting, using a fluorescent antibody diagnostic method, the presence of antibodies of protein constituents of Helicobacter spp. associated with the tissue specimen. Preferably, the invention further includes detecting, using a fluorescent antibody diagnostic method, the presence of virulence factors and toxins protein factors elaborated by the Helicobacter spp. Preferably, the virulence factors are selected from the group consisting of vaculating cytotoxin A, Cag, PAI, iceA, and babA. Preferably, the tissue specimen further includes bodily fluids of said patient. Preferably, the tissue specimen further includes tumor cells of said patient. Preferably, the diagnosed non-gastric disease is selected from the group consisting of carcinoma, sarcoma, mast cell cancer, lymphoma, melanoma, Epuli, diabetes mellitus, gingivitis and periodontitis, peptic and duodenal ulcers or colonization, chronic bowel disease, Crohn's disease, spinal demyelinization and/or multiple sclerosis, Lupus like diseases, degenerative kidney disease, benign prostatic hypertrophy, cardiomyopathy, macular degeneration, retinitis, glaucoma, rheumatoid arthritis, degenerative liver disease, diabetes mellitus (Type I diabetes), periodontal disease and gingivitis, gastroesophageal reflux disease (GERD), and various other immune-mediated diseases. Preferably, the Helicobacter spp. is selected from the group consisting of Helicobacter pylori, Helicobacter heilmannii, Helicobacter fells, Helicobacter mustelae, Helicobacter bizzozeronii, Helicobacter salomonis, Helicobacter canis, Helicobacter cinaedi, Helicobacter fexispira, Helicobacter fennelliae, Helicobacter nemestrinae, Helicobacter dinaedii, Helicobacter pametensis, Helicobacter bilis, and F. R. Rappin.

The invention further includes a method for treating the cause of a diagnosed non-gastric disease in a patient, including: detecting the presence of a Helicobacter spp. associated with the diagnosed non-gastric disease in at least one of bodily tissues and fluids of the patient; and administering a treatment for a duration to the patient. Preferably, the treatment is selected from the group consisting of antibiotic therapy, molecular signal therapy, vaccination, interference with receptor systems mediating Helicobacter spp. invasion into tissues, and use of small-interfering RNA sequences to specifically block virulence or replication of Helicobacter spp. Preferably, the duration is between 60 days and 120 days. Preferably, the duration is 90 days. Preferably, the antibiotic therapy includes administering to said patient an at least one antibiotic selected from the group consisting of metronidazole, Keflex, amoxicillin, tetracycline, clathrimycin, bismuth and the herb, mastic gum or combinations of any of these compounds to the patient. Preferably, the molecular signal therapy includes administering to the patient a molecular component of the Helicobacter spp. Preferably, the molecular component is selected from the group consisting of killed organism, virulence factors, and toxins elaborated by said Helicobacter spp. Preferably, the vaccination includes administering to the patient a molecular constituent of Helicobacter spp. to evoke an immune response by the patient. Preferably, the use of small-interfering RNA sequences to specifically block virulence or replication of Helicobacter spp. includes administering to the patient the small-interfering RNA sequences to regulate the expression of the virulence and toxin factors of the Helicobacter spp. Preferably, the virulence and toxin factors include vacuolating cytotoxin Vac A, Cag PAI, iceA, and babA. Preferably, detecting the presence of the Helicobacter spp. includes analyzing the at least one bodily tissue and fluids of the patent by fluorescent in-situ hybridization analysis.

The invention further includes a test kit for analyzing a tissue specimen of a patient for the presence of unspecified Helicobacter spp. DNA related to a diagnosed non-gastric disease of the patient, including an agent for synthesizing a single strand of a nucleotidic sequence of the Helicobacter spp. DNA of the tissue specimen for binding a fluorescent moiety on the single strand; an agent for hybridizing the DNA nucleotidic sequence for annealing the hybridizing agent to the nucleotidic sequence; and an agent for washing the DNA nucleotidic sequence to remove any unannealed hybridizing agent from the nucleotidic sequence. Preferably, the test kit for analyzing a tissue specimen of a patient further includes an agent to deparaffinize the tissue specimen that is stored in at least one of formalin and paraffin. Preferably, the tissue specimen are tumor cells from said patient.

### BRIEF DESCRIPTION OF THE DRAWINGS

FIG. 1 is a photomicrograph showing stain positive Helicobacter spp. DNA of canine oral tissue infected with gingivitis and periodontitis;

FIG. 2 is a photomicrograph showing stain positive Helicobacter spp. DNA semi-surrounding a feline oral lesional mass of cancer cells;

FIG. 3 is a photomicrograph showing stain positive anti-Helicobacter spp. surrounding human tongue invasive squamous cell carcinoma;

FIG. 4 is a photomicrograph showing stain positive anti-Helicobacter spp. surrounding feline tongue invasive squamous cell carcinoma;

FIG. 5 is a photomicrograph showing stain positive anti-Helicobacter spp. surrounding osteosarcoma in the front leg of a canine;

FIG. 6 is a photomicrograph showing stain positive anti-Helicobacter spp. surrounding canine oral malignant melanoma;

FIG. 7 is a photomicrograph showing stain positive anti-Helicobacter spp. surrounding a malignant melanoma of a feline eyelid;

FIG. 8 is a photomicrograph showing stain positive anti-Helicobacter spp. highlighting a Krukenberg tumor comprising a cluster of cancer cells in human ovary tissue;

FIG. 9 is a photomicrograph showing stain positive enlarged single cluster of Helicobacter spp. DNA and anti-Helicobacter spp. of FIG. 8;

FIG. 10 is a photomicrograph showing stain positive anti-Helicobacter spp. surrounding adenocarcinoma in the stomach of a canine;

FIG. 11 is a photomicrograph showing stain positive anti-Helicobacter spp. surrounding adenocarcinoma in the stomach of a human;

FIG. 12 is a photomicrograph showing stain positive anti-Helicobacter spp. surrounding and intermixed with cardiac anterior left ventricle muscle cells suffering from myocardial degeneration of a human;

FIG. 13 is a photomicrograph showing stain positive anti-Helicobacter spp. and anti-Helicobacter DNA in human pancreas cells;

FIG. 14 is a photomicrograph showing stain positive Helicobacter DNA and anti-Helicobacter spp. intermixed with unaffected spinal tissue of a canine affected by spinal demyelinization;

FIG. 15A is a photomicrograph showing uptake of an anti-Helicobacter antibody fluorescent reagent stain of a ileal-colic intestinal lining of a canine;

FIG. 15B is a photomicrograph showing uptake of an anti-Helicobacter antibody fluorescent reagent stain of a spleen of a canine spleen;

FIG. 16 is a photomicrograph at 100× microscopic magnification showing uptake of an anti-Helicobacter antibody fluorescent reagent stain for Helicobacter spp. of a biopsy of oral tissue of a human;

FIG. 17 is a photomicrograph at 100× microscopic magnification showing uptake of an anti-Helicobacter antibody fluorescent reagent stain for Helicobacter spp. of a biopsy of oral tissue of a feline;

FIG. 18A is a photomicrograph at 100× microscopic magnification showing uptake of an Helicobacter DNA fluorescent reagent stain for Helicobacter spp. of a biopsy of eye tissue of a human;

FIG. 18B is a photomicrograph at 50× microscopic magnification showing uptake of an Helicobacter DNA fluorescent reagent stain for Helicobacter spp. of a biopsy of eye tissue of a human;

FIG. 19 is a photomicrograph at 100× microscopic magnification showing uptake of an anti-Helicobacter antibody fluorescent reagent stain for Helicobacter spp. of a biopsy of eye tissue of a human;

FIG. 20 is a photomicrograph at 100× microscopic magnification showing uptake of an anti-Helicobacter spp. VAC protein (Toxin) antibody fluorescent reagent stain for Helicobacter spp. of a biopsy of eye tissue of a human;

FIG. 21 is a photomicrograph at 50× microscopic magnification showing uptake of an anti-Helicobacter spp. CAG antigen fluorescent reagent stain for Helicobacter spp. of a biopsy of eye tissue of a human;

FIG. 22 is a photomicrograph at 100× microscopic magnification showing uptake of an Helicobacter spp. antibody fluorescent reagent stain for Helicobacter spp. of a biopsy of eye tissue of a canine;

FIG. 23 is a photomicrograph at 100× microscopic magnification showing uptake of an anti-Helicobacter spp. antibody fluorescent reagent stain for Helicobacter spp. of a biopsy of eye tissue of a canine;

FIG. 24 is a photomicrograph at 100× microscopic magnification showing uptake of an Helicobacter spp. DNA fluorescent reagent stain for Helicobacter spp. of a biopsy of eye tissue of a canine; and

FIG. 25 is a photomicrograph at 100× microscopic magnification showing uptake of an anti-Helicobacter spp. antibody fluorescent reagent stain for Helicobacter spp. of a biopsy of eye tissue of a canine.

### DETAILED DESCRIPTION OF THE DRAWINGS

In accordance with the present method of diagnosis, treatment, and vaccination of Helicobacter spp. associated infections (“present invention”), the present invention provides molecular detection of various components of the Helicobacter spp. organisms within bodily tissues and fluids. Some exemplary Helicobacter spp. include: Helicobacter (H.) pylori, H. heilmannii, H. fells, H. mustelae, H. bizzozeronii, H. salomonis, H. canis, H. cinaedi, H. fexispira, H. fennelliae, H. nemestrinae, H. dinaedii, H. pametensis, H. bilis, F. R. Rappini, and other species yet to be identified or described in the literature. These methods of diagnosis are specific to Helicobacter organisms and do not exhibit cross reactivity with components of other pathogens or endogenous bodily structures. Such diagnostic tests also possess sufficient sensitivity to detect the presence of relatively low levels of individual pathogens.

One of the preferred methods of the present invention is the detection of specific gene sequences through the use of fluorescent in-situ hybridization (FISH), a method well known in the art (Trebesius, et al, 2000). As used herein, a gene sequence is a linear chain of any length comprised of the nucleotides: cytosine (C), adenine (A), guanosine (G), or tyrosine (T). An exemplary method includes the use of the following DNA hybridization probe: 5′-CAC-ACC-TGA-CTG-ACT-ATC-CCG-3′ (Sequence ID number 1). Another exemplary method includes the use of the following DNA hybridization probe: 5′-GCC-GTG-CAG-CAC-CTG-TTT-TCA-3′ of the nucleotidic sequence.

Generally, the FISH technique detects target molecules with a system of antibodies and fluorochromes. The detection of nucleotidic sequences on a specific DNA is performed by first hybridizing the nucleotidic sequences with the DNA. Sequence ID number 1 is specific to a sequence located within the 16S ribosomal RNA gene of Helicobacter Pylor. The nucleotidic sequences are synthesized with incorporated fluorescent or antigenic sites, which can be recognized with fluorescent antibodies. Sequences designed for use in FISH detection procedures are labeled for detection by a variety of fluorescent moieties including, but not limited to, FITC, TRITC, Texas red, and rhodamine allowing localization of the binding site within histological. sections by excitation of the fluorescent conjugate at specific incident light wavelengths resulting in emission of light at longer wavelengths. Such fluorescent probes may be attached to the DNA binding sequence in a variety of ways well known to those skilled in the art including, conjugation to the 5′ end of the hybridizing sequence. Conjugation methods are designed to covalently attach fluorescent moieties to the DNA binding sequence while at the same time preserving the ability of the DNA probe to hybridize with complementary sequences located within the tissue specimen. Preferably, a single strand of the double strand DNA nucleotidic sequences is synthesized by the use of a polymerase enzyme to incorporate a labeled nucleotides onto the single strand, which is followed by denaturation.

Preferably, denaturation is obtained by heating the DNA, which separates the two strands and allows access of the single strand probes to their respective nucleotidic sequences. Finally, the detection of the nucleotidic sequences is performed by recognizing the fluorescent antibodies corresponding to the antigens incorporated into the nucleotidic sequences. Preferably, the detection is done with a epifluorescence microscope. Typically, the white light of the lamp source is filtered so that only the desired wavelengths of light are emitted for excitation of the fluorescent molecules. Generally, the emissions of fluorochromes occur at a longer wavelength, which allows the distinction between the excitation wavelengths of the optical sources and the emission wavelengths. Additional filters or filter sets may be used to further distinguish between several excitation and emission wavelength bands.

The present invention includes additional DNA sequences for use in the FISH procedure for diagnostic detection of Helicobacter spp. organisms. Such sequences are operationally defined according to the specific characteristics of the DNA sequence and include the following: Helicobacter species-specific sequences, genus-specific sequences common to all or several Helicobacter spp., altered sequences that do not bind to Helicobacter target sequences, and sequences binding to antibiotic resistant strains of Helicobacter spp.

Irrespective of the exact DNA probe composition or its attached fluorescent marker, a common method is used to perform the FISH procedure involving preparation of tissue sections, hybridization, (with or without multiple labeling and counterstaining) followed by detection. These are standard procedures well known to those skilled in the art. In addition, the present invention is intended to broadly encompass FISH procedures that include hybridization of fluorescent conjugated DNA probes to tissue sections, localization of such binding sites within specific regions of the histological or cytological specimen, and the diagnostic determination of the presence or absence of Helicobacter spp. organisms. The present invention is not limited by methodological details used to perform FISH diagnostic procedures.

Another embodiment of the present invention includes diagnostic determination of the presence of Helicobacter spp. molecular constituents within bodily tissues and fluids by immunological methods. A defining characteristic of the immunological procedures associated with the present invention is specificity to Helicobacter spp. components and sensitivity to low levels of such constituents. One such method includes the use of fluorescent-labeled antibodies or secondary antibodies in conjunction with FISH diagnostic procedures outlined above. A fluorescent antibody diagnostic method involves use of antibodies specific to protein constituents of Helicobacter spp. including the organism itself and/or protein factors elaborated by the organism including virulence factors and toxins involved in the invasion of Helicobacter spp. into its host. While antigen specificity is implied, the invention also encompasses multiple determinant antibodies reacting to a mixture of the above-defined constituents. The presence of reactive antigens with tissues may be determined by suitable incubation with the antibody and detection of antigen localization through methods well known in the art. One such method involves use of a fluorescent-conjugated secondary antibody and is described in Example 1 below. However, the invention broadly encompasses immunological detection of Helicobacter spp.-specific molecular components within bodily tissues and is not limited by specific methodological details. The presence of Helicobacter spp.-specific antigenic determinants within tissues is diagnostic of the presence of Helicobacter spp. infection. Such a determination involves consideration of relevant evidence including use of control conditions well known to those skilled in the art.

Immunological diagnostic methods of the present invention also include use of immunological methods to detect Helicobacter spp. components or host-derived Helicobacter spp. specific components within bodily fluids. Such methods may be directed towards any Helicobacter spp. specific molecular component or host-derived component including, but not limited to, antibodies to Helicobacter spp. More preferably, the present invention relates to immunological based measurement of virulence factors and toxins elaborated by Helicobacter spp. as it invades host tissues. Such factors include most preferably vacuolating cytotoxin A but also include Cag PAI, iceA, babA and other Helicobacter spp. virulence factors yet to be identified. Since these virulence factors mediate Helicobacter spp. invasion of tissues and are secreted from tissue-localized organisms, their presence within bodily fluids reflects the extent of Helicobacter spp. invasion beyond the normal presence within the GI tract. Immunoassay of virulence factors by the present invention includes methods known in the art for quantitative and qualitative immunoassay including multiple antibody-based assays and high sensitivity detection systems including, but not limited to, those based on chemi-luminescence.

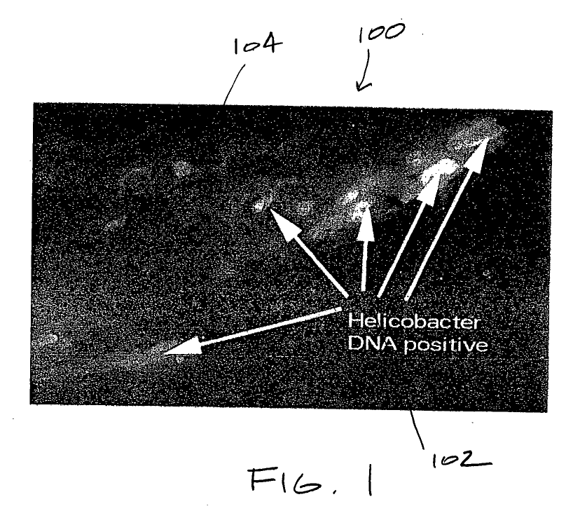

FIG. 1 is a photomicrograph 100 at 100× microscopic magnification showing DNA specific positive tag on Helicobacter spp. organism 102 located within canine oral tissue 104 suffering from gingivitis and periodontitis. The bright areas of the electron micrograph are the fluorescent stain that is attracted to the Helicobacter DNA. The specimen was subjected to a light source excitation wavelength of 494 nm and had an emission wavelength of ≧515 nm that induced fluorescence of the FITC conjugate coupled to the Helicobacter spp.-specific DNA probe.

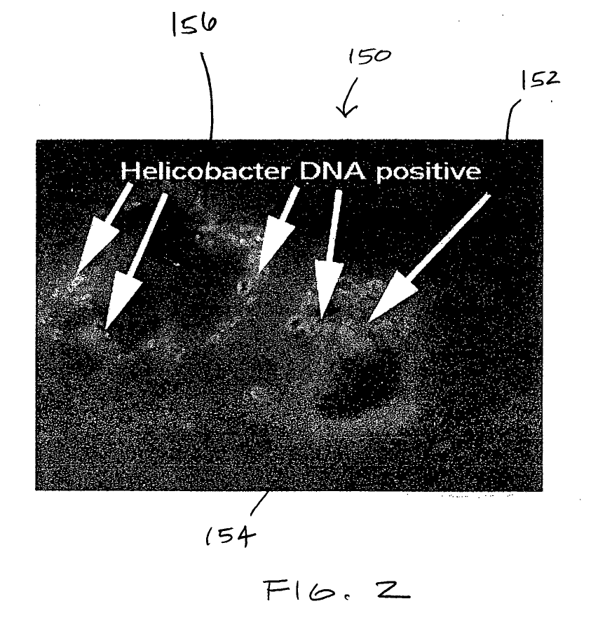

FIG. 2 is a photomicrograph 150 at 100× microscopic magnification showing DNA specific positive tag on Helicobacter spp. organism located within two masses of malignant cancer cells 152 and 156 found in feline oral tissues associated. The cancer cells 152 (larger) and 156 (smaller) are the two clusters where the central dark masses 154 are nuclear material and the bright, white, circular appearing objects are the DNA specific positive tag located within the surrounding Helicobacter spp. DNA. The specimen was subjected to a light source excitation wavelength of 494 nm that induced fluorescence and emission at ≧515 nm which induces fluorescence of the FITC conjugate coupled to the Helicobacter spp.-specific DNA probe surrounding the feline oral lesional mass of cancer cells.

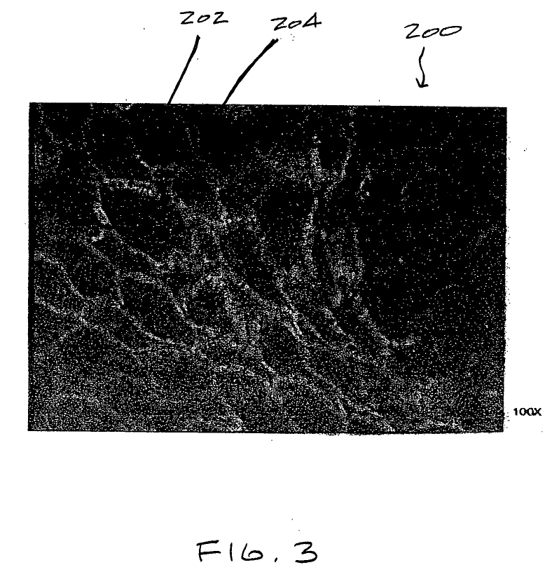

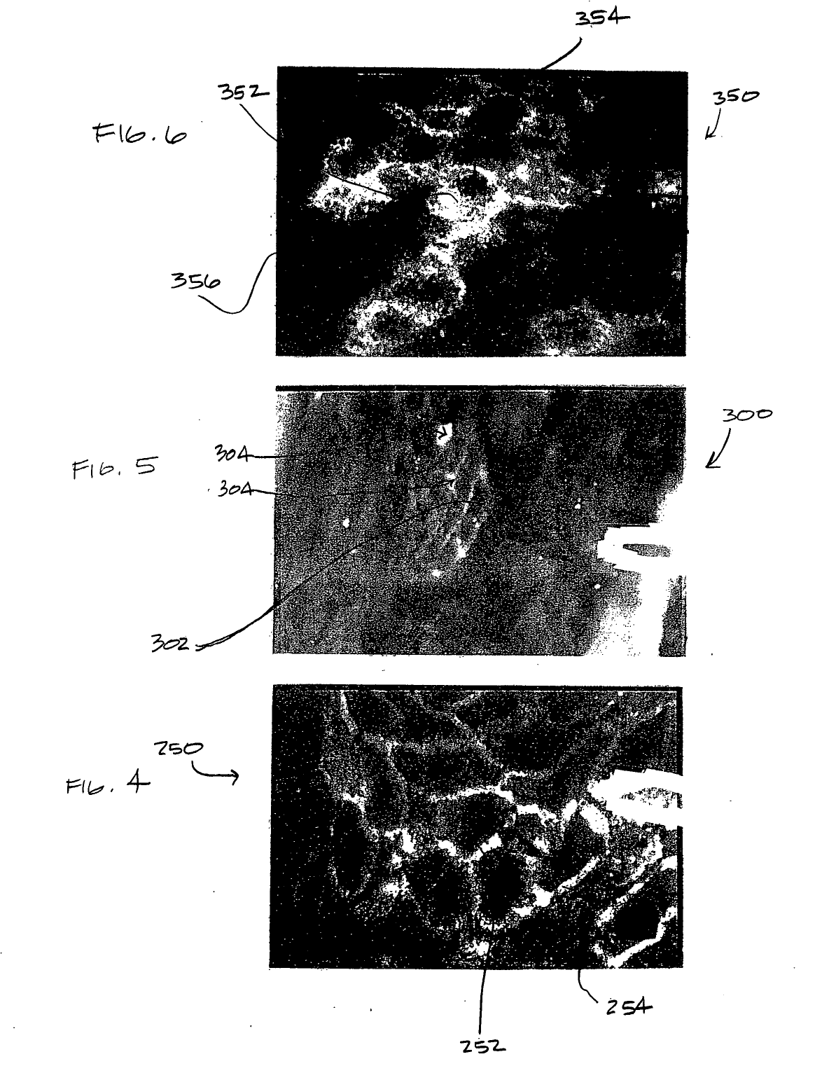

FIG. 3 is a photomicrograph 200 at 100× microscopic magnification showing DNA specific positive tag on Helicobacter spp. DNA of squamous cell carcinoma (malignant) cells from the oral tissue cells (tongue) of a human. The Helicobacter DNA specific reagent 202 uptake is involved with all the cancer cells 204 seen in this tissue section of oral cancer. Additionally, the morphological appearance of this cancer 204 appears identical to that of the feline with the same type of cancer as in FIG. 4. As can be seen from FIG. 3, several squamous cell carcinoma 204 are shown surrounded by the anti-Helicobacter antibody 202. FIG. 3 shows the presence of anti-Helicobacter spp. in a human tongue associated with invasive squamous cell carcinoma 204.

FIG. 4 is a photomicrograph 250 at 100× microscopic magnification showing DNA specific positive tag on Helicobacter spp. DNA on squamous cell carcinoma (malignant) from the oral tissue cells (tongue) of a feline (cat). The Helicobacter DNA specific reagent 252 uptake is involved with all the cancer cells 254 seen in this tissue section of oral cancer. It is important to note that the morphological appearance of this cancer 254 appears identical to that of the human with the same type of cancer as in FIG. 3.

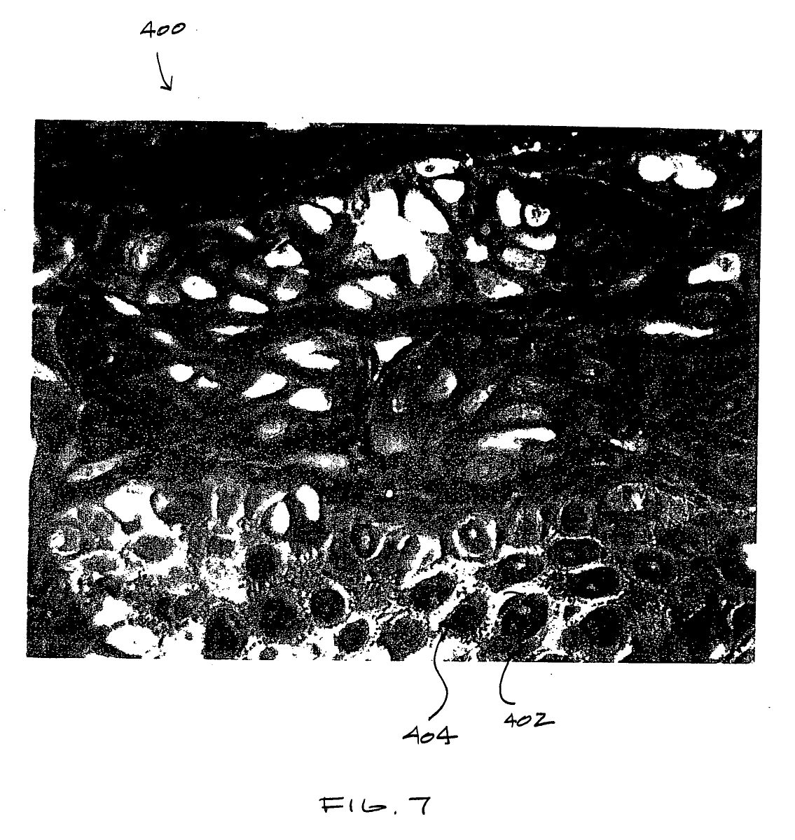

FIG. 5 is a photomicrograph 300 at 100× microscopic magnification showing DNA specific positive tag on Helicobacter spp. DNA on an osteogenic sarcoma (malignant) cells 302 of a femur (leg bone tissue) of a canine. The Helicobacter DNA specific reagent 304 uptake is involved with all cancer cells 302 seen in this tissue section of the osteosarcoma cells with a dark central nuclear mass which contains no Helicobacter.

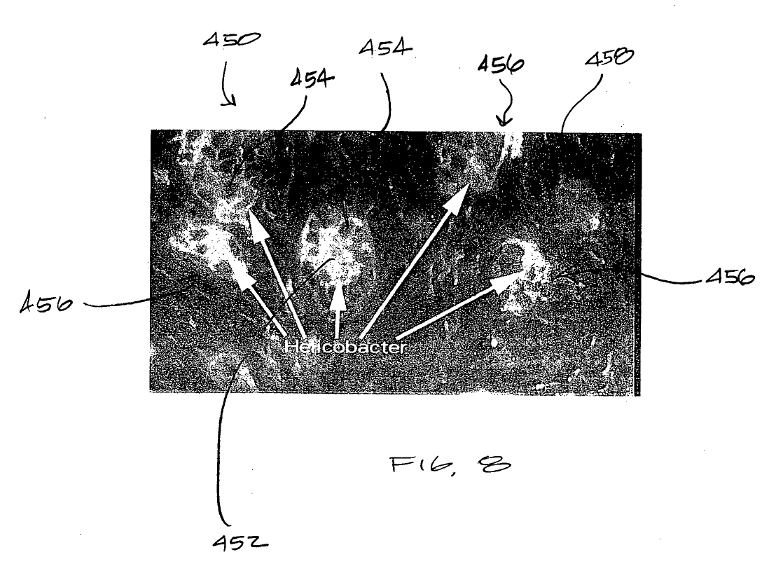

FIG. 6 is a photomicrograph 350 at 100× microscopic magnification showing DNA specific positive tag on Helicobacter spp. DNA on a malignant melanoma (malignancy) cells 352 of a canine tongue primary lesion. The Helicobacter DNA specific reagent 352 uptake is involved with all the cancer cells 352 seen in this tissue section of malignant melanoma cells with a dark central nuclear mass which contains no Helicobacter. Also, surrounding normal tissues of the tongue show no Helicobacter specific DNA reagent uptake.

FIG. 7 is a photomicrograph 400 at 100× microscopic magnification showing DNA specific positive tag on Helicobacter spp. DNA on a malignant melanoma (malignancy) cells 402 of a feline (cat) eye lid tumor primary lesion. The Helicobacter DNA specific reagent 402 uptake is involved with the cancer cells 402 seen in this tissue section at the bottom edge of the photomicrograph where it is seen that a dark central nuclear mass 404 which contains no Helicobacter. Also, surrounding normal tissues of the tongue show no Helicobacter specific DNA reagent uptake. A triple stain was utilized on this tissue examination by which two molecules wre also demonstrated: CAG and Vac-A (cytotoxin).

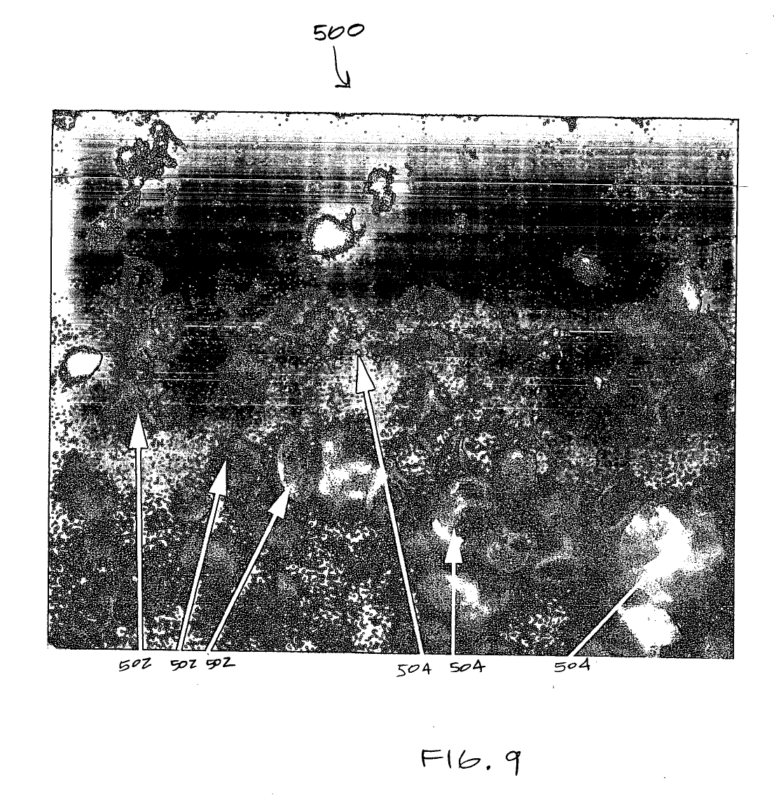

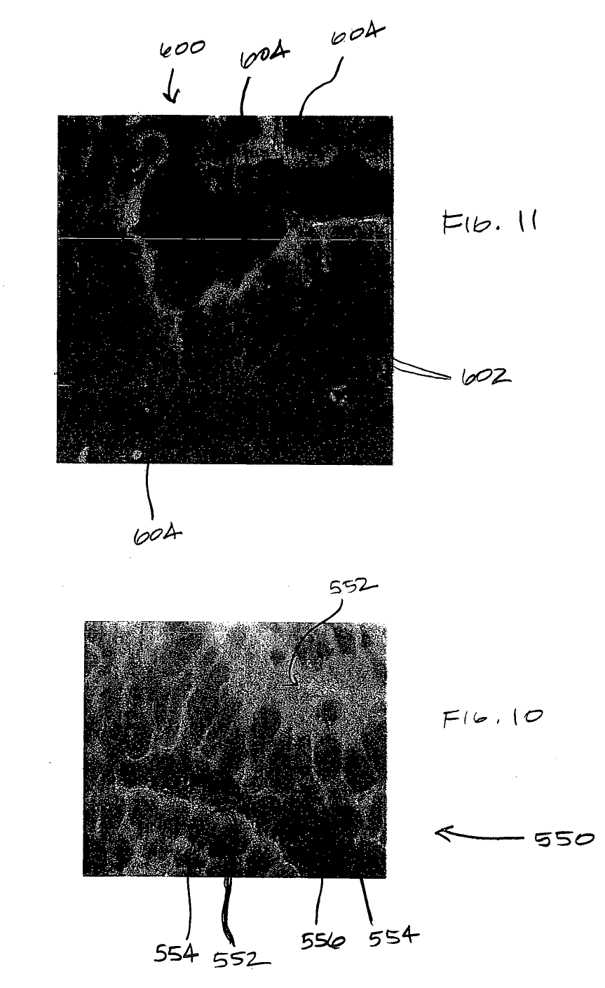

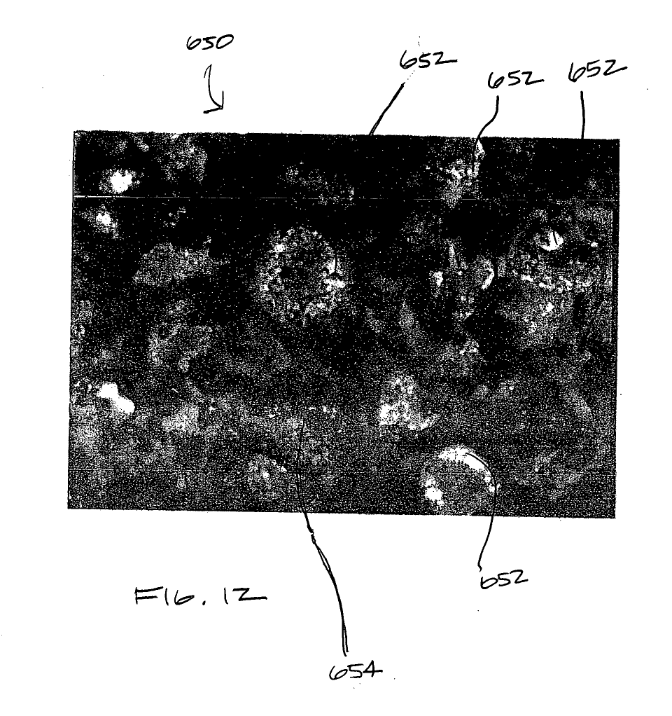

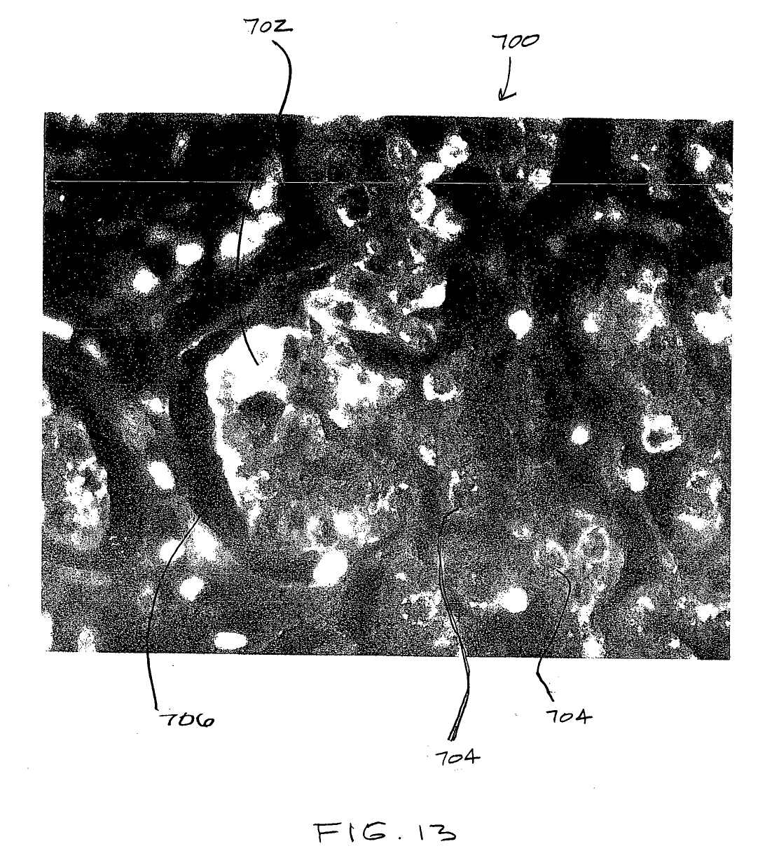

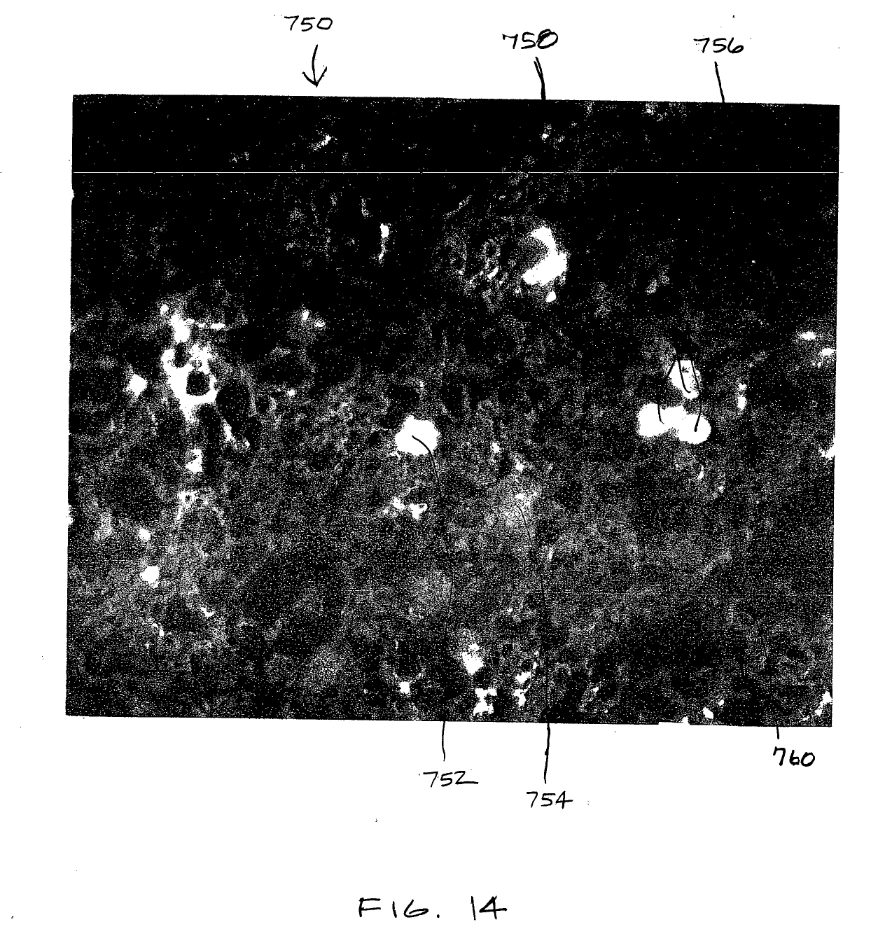

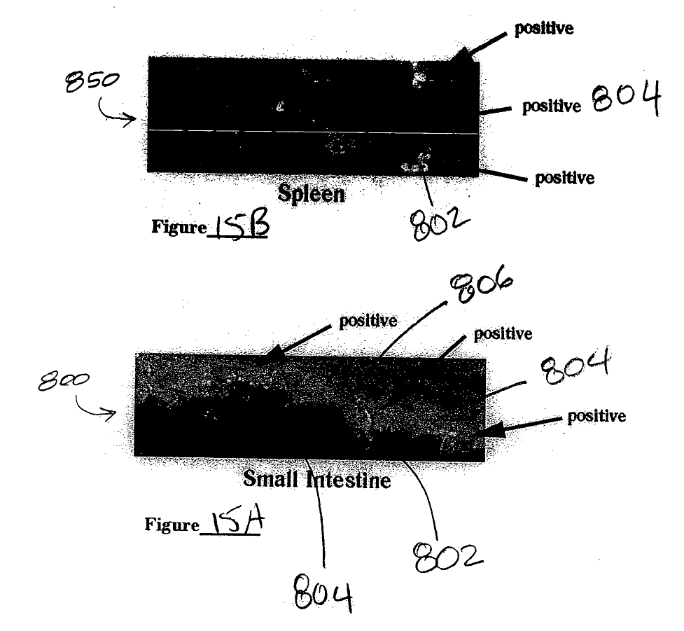

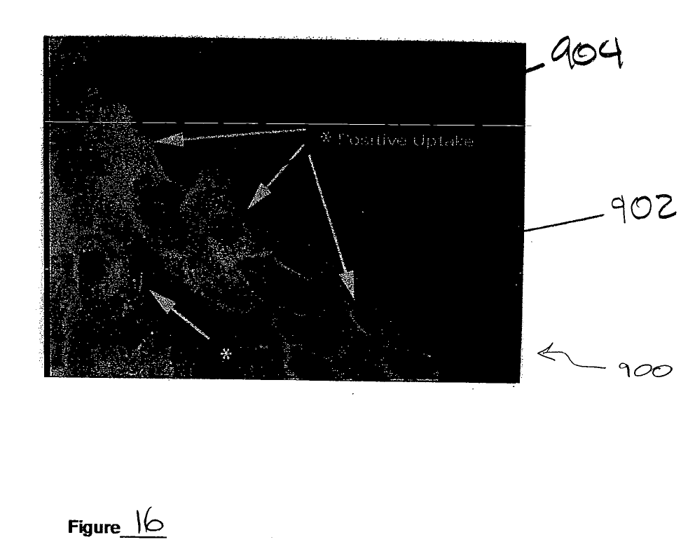

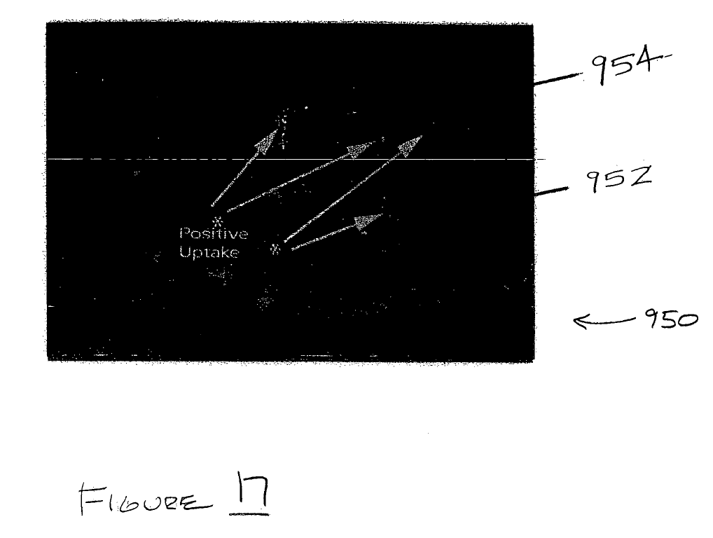

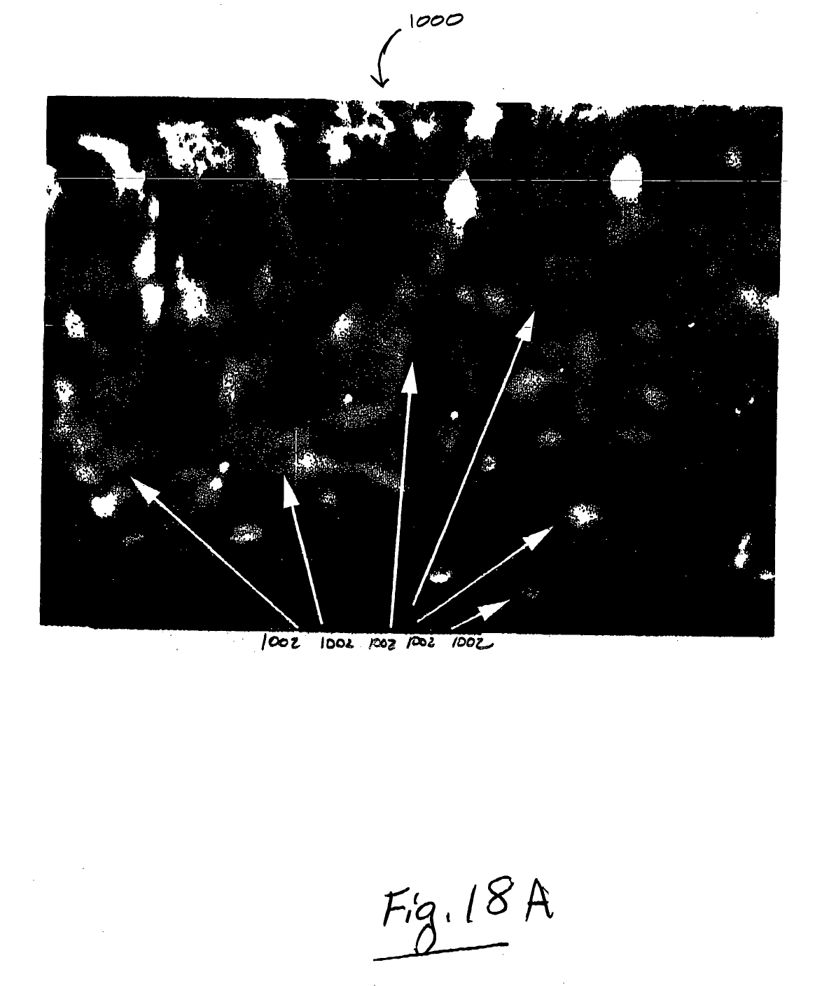

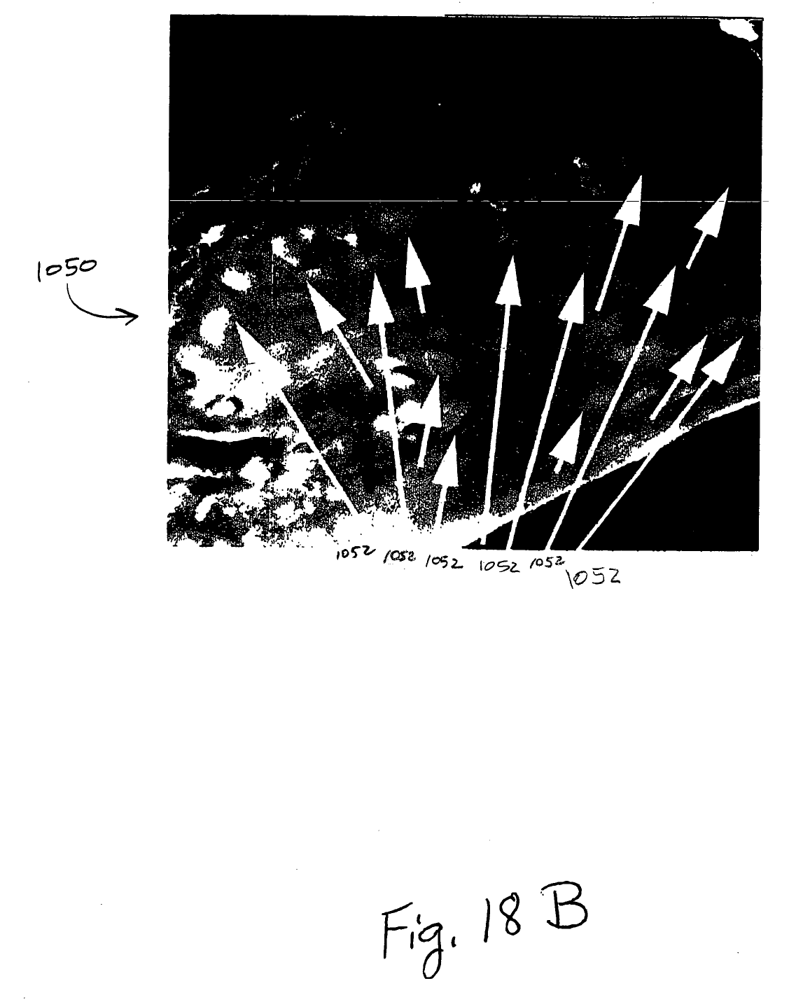

FIG. 8 is a photomicrograph 450 showing uptake of an anti-Helicobacter antibody fluorescent reagent stain for Helicobacter spp. Island (colonies) of adenocarcinoma cells 452 containing Helicobacter spp. identified in the human ovary which is classified as a a Krukenberg tumor in this case, human ovarian cancer associated with adenocarcinoma of the stomach, at 100× microscopic magnification. This technique identified the present of Helicobacter antigen 456 associated with the adenocarcinoma cells 452 in the ovary of a human with ovarian Krukenberg tumor. The island of cancer cells 452 are clearly identified and it is notable that the normal ovarian cells 458 between these islands do not uptake the antibody reagent.

FIG. 9 is a photomicrograph 500 showing DNA specific positive tag on Helicobacter spp. DNA on a cluster (colony) of adenocarcinoma cells of a human Krukenberg ovarian cancer 502 uptake is seen around the cancer cells seen in this tissue section at the bottom edge of the photomicrograph where it is seen that a dark central nuclear mass 504 which contains no Helicobacter. Also, surrounding normal tissues of the tongue show no Helicobacter specific DNA reagent uptake. A triple stain was utilized on this tissue examination by which two molecules were also demonstrated: CAG and Vac-A (cytotoxin).

FIG. 10 is a photomicrograph 550 at 100× microscopic magnification showing uptake of an anti-Helicobacter antibody fluorescent reagent stain for Helicobacter spp. This is a cross section of an adenocarcinoma of the stomach of a canine (dog) where the Helicobacter organisms are tagged in the lumen of the stomach 556 and the adenocarcinoma cancer tissues in the wall of the stomach 552 are seen to possess Helicobacter spp. antigens in the tumor tissue 552. It is observed that the dark central areas 554 of the cancer cells reveal no Helicobacter uptake in the areas of cell nucleus.

FIG. 11 is a photomicrograph 600 at 100× microscopic magnification showing uptake of an anti-Helicobacter antibody fluorescent reagent stain for Helicobacter spp. This is a cross section of an adenocarcinoma tumor in the stomach wall of a human where the Helicobacter organisms are tagged 602 and are seen to possess Helicobacter spp. antigens in the tumor tissue 602. it is observed that the dark central areas 604 of the cancer cells reveal no Helicobacter uptake in the areas of cell nucleus. The similarity of canine (dog) of FIG. 10 and human stomach cancer with Helicobacter spp. involvement is noteable.

FIG. 12 is a photomicrograph 650 at 100× microscopic magnification showing uptake of an anti-Helicobacter antibody fluorescent reagent stain for Helicobacter spp. This is a cross section of a live biopsy of cardiac tissue of a human heart muscle, anterior left ventricle, from a patient with cardiomyopathy who demonstrates PVC's. The cross section of heart muscle demonstrates infiltration by Helicobacter spp. into the myocardial tissues 652 where as adjacent tissues, normal myocardial tissues 654, demonstrate no uptake nor infiltration. The significance of this observation is that Helicobacter is identified as an etiological agent in the process of myocardial degeneration and it may be supposed from this observation and current observations concerning the presence of CAG (immuno-suppression) and Vac-A (cytotoxin) that this organism is of great importance in the mechanism of pathology in myocardial degenerative conditions.

FIG. 13 is a photomicrograph 700 at 100× microscopic magnification showing DNA specific positive tag on Helicobacter spp. DNA of a human pancreatic Islet from a patient who died of diabetes mellitus. The Helicobacter DNA specific reagent 704 uptake is seen throughout the pancreas with a virtual absence of morphologically identifiable Beta cells. A triple stain 706 was utilized on this tissue examination by which two molecules were also demonstrated: CAG and Vac-A (cytotoxin). The significance of this observation is that Helicobacter is identified as an etiological agent in the mechanism of Islet/Beta cell destruction in the pancreas leading to diabetes mellitus. This observation has also been demonstrated in canine (dog) and feline (cat) patients with diabetes mellitus.

FIG. 14 is a photomicrograph 750 showing DNA specific positive tag on Helicobacter spp. DNA at 100× microscopic magnification of canine (dog) spinal cord tissue taken postmortem from a patient who died and was afflicted with spinal cord demyelinization—a condition similar to or the same as multiple sclerosis in humans. The Helicobacter DNA specific reagent 752, 754, 756 uptake is seen scattered throughout the spinal cord cross section. Where there is no DNA specific reagent uptake 760 it is notable that this patient still retained functions of the spinal cord, extreme paresis and loss of proprioception being the primary symptoms. A double stain was utilized on this tissue examination. The significance of this observation is that Helicobacter is identified as an etiological agent in the mechanism of canine spinal cord demyelinization and possibly human multiple sclerosis. This photomicrograph 750 shows that Helicobacter as an agent of IBD and Crohn's Disease. Present day treatments for Crohn's disease involves administering steroids to the patient, generally followed by chemotherapy and then possibly surgery. Even with these protocols, the patent may not survive the disease. The present invention would detect the presence of the Helicobacter organisms, which could then be treated by antibiotics that would destroy the Helicobacter organism, thus destroying the disease. Helicobacter antibody 758 is shown.

FIG. 15A is a photomicrograph 800 at 100× microscopic magnification showing uptake of an anti-Helicobacter antibody fluorescent reagent stain for Helicobacter spp. This is a cross section of canine ileal-colic intestinal lining of FIG. 15A and canine spleen 850 of FIG. 15B. The cross section of canine intestine showed the presence of Helicobacter spp. organism and antigen 802, 804, and 806 through the affected tissues found in this case of ‘intestinal disease’ which is similar to or the same as human Crohn's disease. The lumen of the intestine demonstrated no Helicobacter by the spleen appeared to be heavily seeded with the organism 802 and 804. The significance of this observation is that Helicobacter is identified as an etiological agent in the process of ileal-colonic diseases of the canine and possibly the human (Crohn's disease) and it may be supposed from this observation and current observations concerning the presence of CAG (immuno-suppression) and Vac-A (cytotoxin) that this organism is of great importance in the mechanism of pathology in these inflammatory/degenerative disease conditions.

FIG. 16 is a photomicrograph 900 at 100× microscopic magnification showing uptake of an anti-Helicobacter antibody fluorescent reagent stain for Helicobacter spp. This is a biopsy of oral tissues (mouth) of a human with gingivitis and periodontal disease. This section of gingival tissue shows the presence of Helicobacter spp. organism and antigen 902 and 904 through the affected tissues found in this case. The examination of canine and feline gingival tissues from patients with gingivitis and periodontal disease are near identical in appearance with no Helicobacter but the spleen appeared to be heavily seeded with the organism 902 and 904. The significance of this observation is that Helicobacter is identified as an etiological agent in the process of gingivitis and periodontal disease, noting that in areas of healthy adjacent tissues there is evidence of Helicobacter spp. and it may be supposed from this observation and current observations concerning the presence of CAG (immuno-suppression) and Vac-A (cytotoxic) that this organism is of great importance in the mechanism of pathology in these inflammatory/degenerative disease conditions such as gingivitis and periodontal disease in cats, dogs, and humans.

FIG. 17 is a photomicrograph 950 at 100× microscopic magnification showing uptake of an anti-Helicobacter antibody fluorescent reagent stain for Helicobacter spp. at 100× microscopic magnification. This is a biopsy of oral tissue (mouth) of a feline (cat) with a squamous cell carcinoma (malignant). The nucleus of the cancer cells 954 did not uptake the Helicobacter antibody reagent stain but the cancer cells themselves appeared to have a compliment of organisms 952 on the cell surface. The significance of this observation is that Helicobacter is identified as being associated with squamous cell carcinoma of the oral cavity similarly to observations of adenocarcinoma of the stomach (cancer) of an etiological agent in the process of gingivitis and periodontal disease, noting that in areas of healthy adjacent tissues there is evidence of Helicobacter spp. and it may be supposed from this observation and current observations concerning the presence of CAG (immuno-suppression) and Vac-A (cytotoxic) tht this organism is of great importance in the mechanism of pathology in these inflammatory/degenerative disease conditions such as gingivitis and periodontal disease in cats, dogs, and humans.

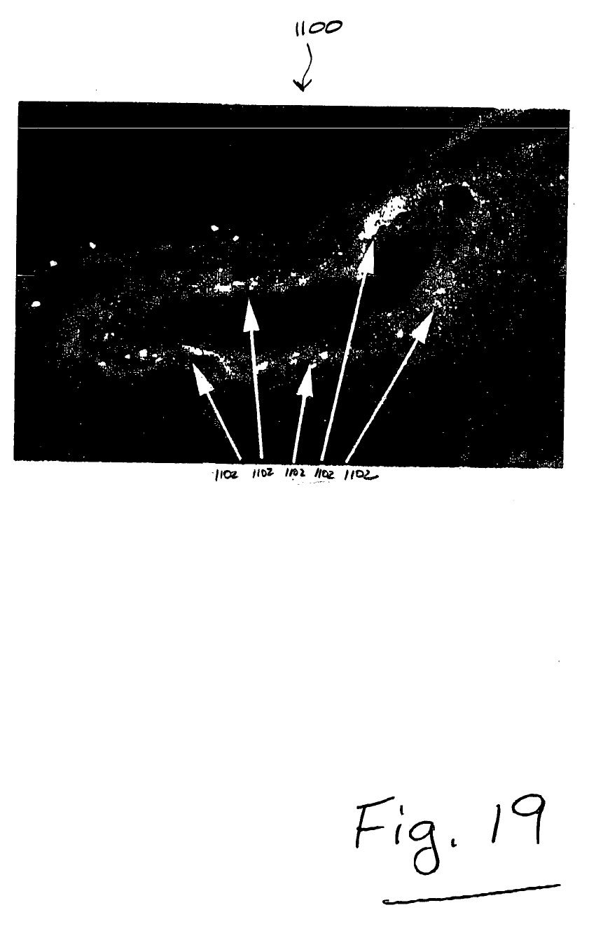

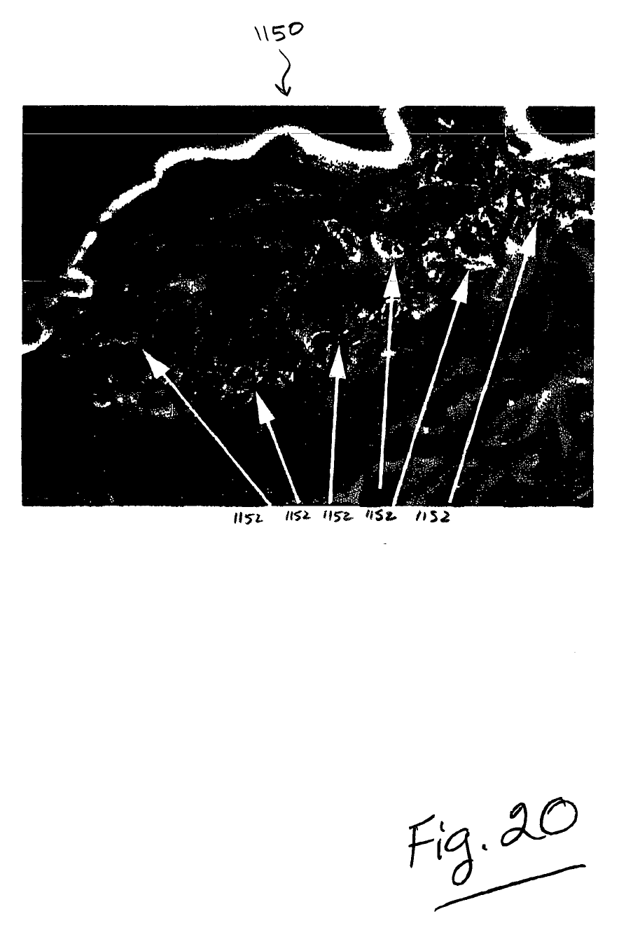

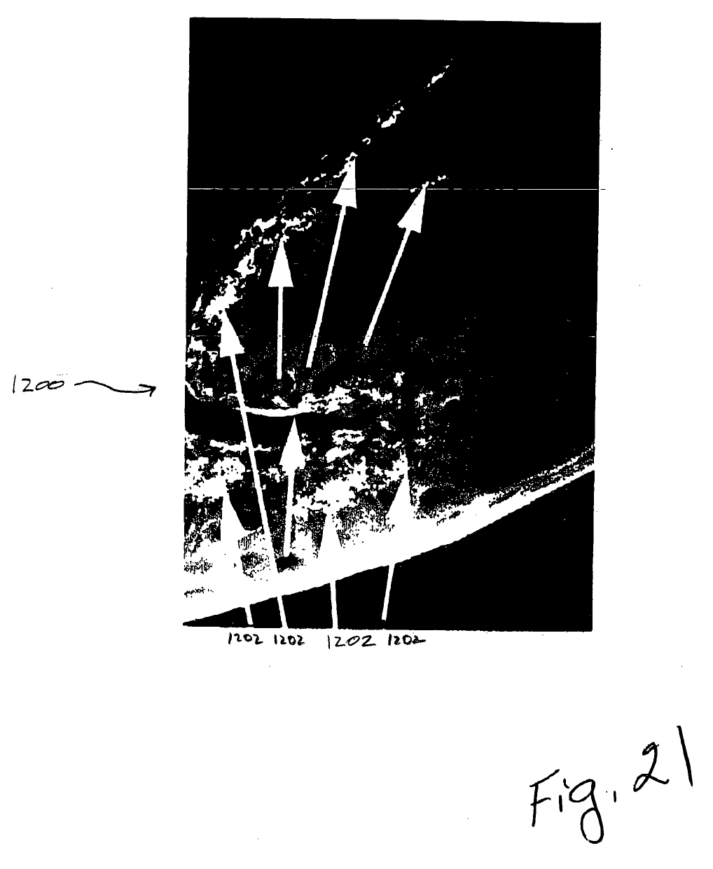

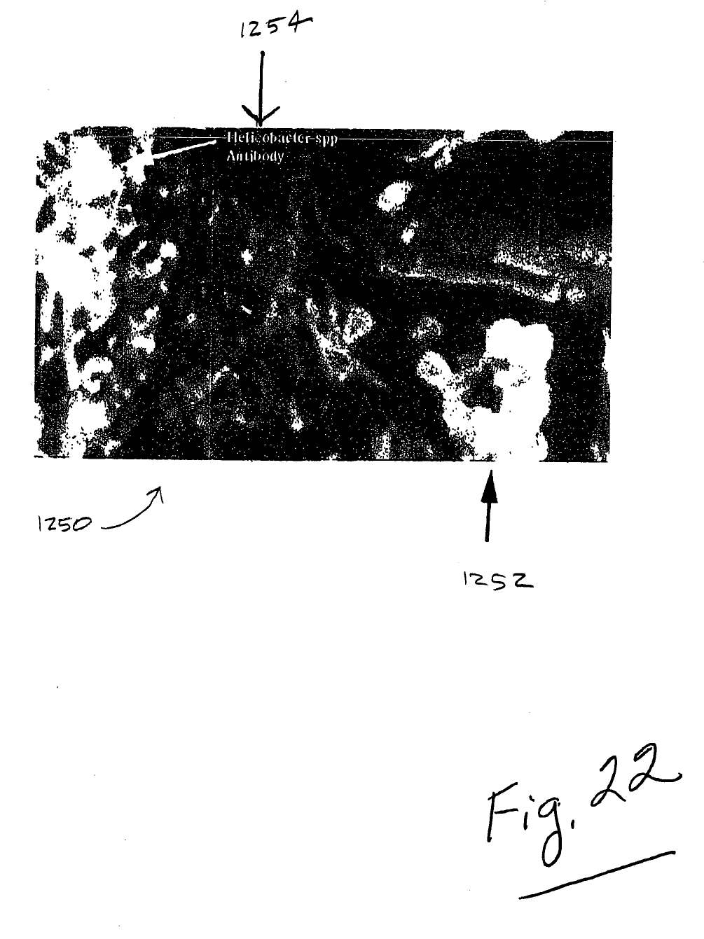

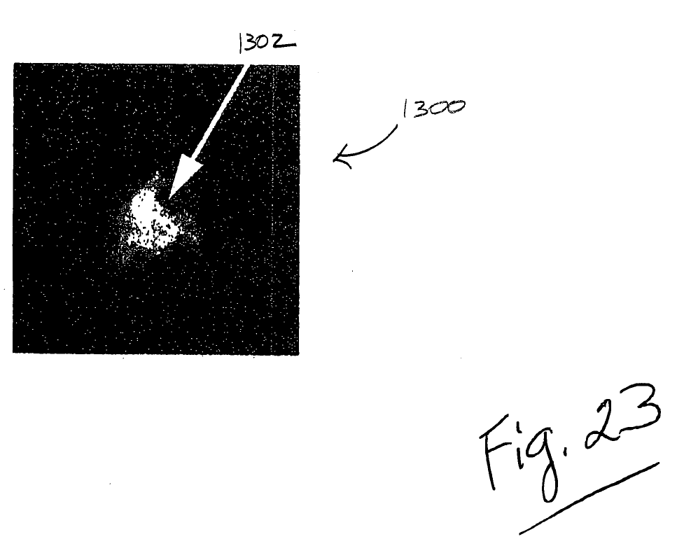

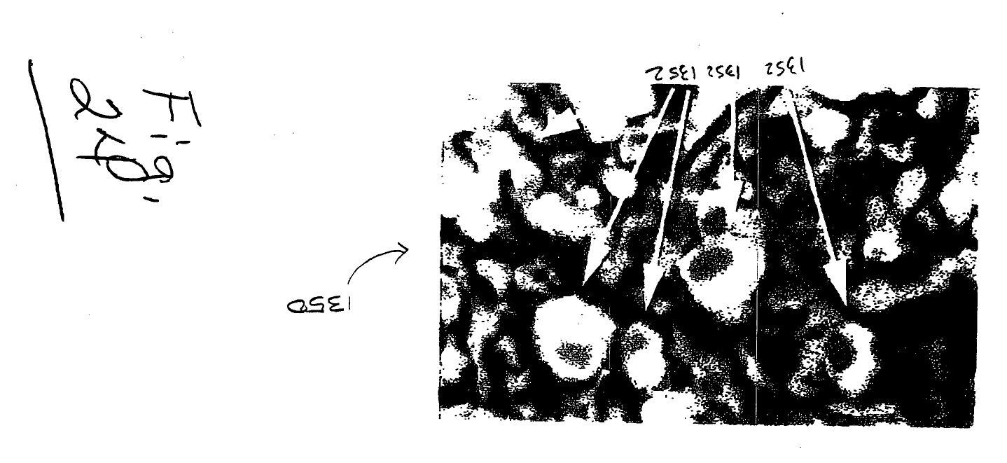

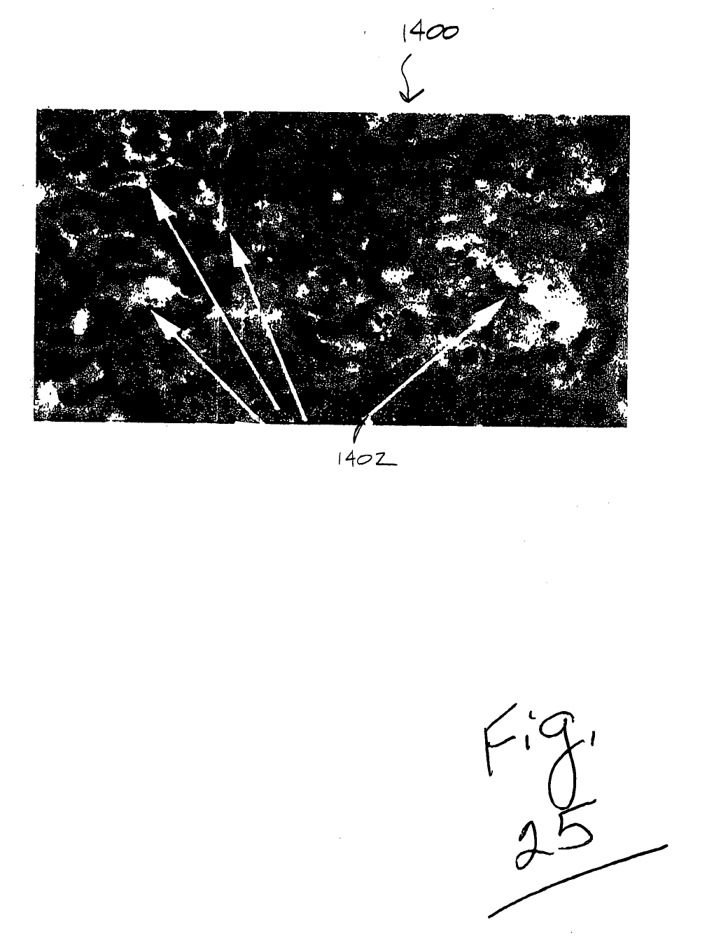

FIG. 18A is a photomicrograph 1000 at 100× microscopic magnification showing uptake of an Helicobacter DNA fluorescent reagent stain 1002 for Helicobacter spp. This is a biopsy of eye tissue of a human with macular degeneration. FIG. 18B is a photomicrograph 1050 at 50× microscopic magnification showing uptake of an Helicobacter DNA fluorescent reagent stain 1052 for Helicobacter spp. This is a biopsy of eye tissue of a human with macular degeneration. FIG. 19 is a photomicrograph 1100 at 100× microscopic magnification showing uptake of an anti-Helicobacter antibody fluorescent reagent stain 1102 for Helicobacter spp. This is a biopsy of eye tissue of a human with macular degeneration. FIG. 20 is a photomicrograph 1150 at 100× microscopic magnification showing uptake of an anti-Helicobacter spp. VAC protein (Toxin) antibody fluorescent reagent stain 1152 for Helicobacter spp. This is a biopsy of eye tissue of a human with macular degeneration. FIG. 21 is a photomicrograph 1200 at 50× microscopic magnification showing uptake of an anti-Helicobacter spp. CAG antigen fluorescent reagent stain for Helicobacter spp. This is a biopsy of eye tissue of a human with macular degeneration. FIG. 22 is a photomicrograph 1250 at 100× microscopic magnification showing uptake of an Helicobacter spp. antibody fluorescent reagent stain 1252 and anti-Helicobacter spp. antibody for Helicobacter spp. This is a biopsy of eye tissue of a canine with glaucoma. FIG. 23 is a photomicrograph 1300 at 100× microscopic magnification showing uptake of an anti-Helicobacter spp. antibody fluorescent reagent stain 1302 for Helicobacter spp. This is a biopsy of eye tissue of a canine with glaucoma. FIG. 24 is a photomicrograph 1350 at 100× microscopic magnification showing uptake of an Helicobacter spp. DNA fluorescent reagent stain 1352 for Helicobacter spp. This is a biopsy of eye tissue of a canine with retinitis. FIG. 25 is a photomicrograph 1400 at 100× microscopic magnification showing uptake of an anti-Helicobacter spp. antibody fluorescent reagent stain 1402 for Helicobacter spp. This is a biopsy of eye tissue of a canine with retinitis.

In one embodiment, specimens, such as those described in FIGS. 1-25, may be prepared according to the following preparation technique. Generally, tissue from an infected area of a patient that exhibits symptoms is extracted by a health professional. In one aspect, the tissue is then fixed in 10% formalin and then 2-micron thick sections are prepared from paraffin-embedded material by standard methods. Preferably, the tissue sections are then spotted and fixed onto glass slides by incubation overnight at 55° C., and then deparaffinized by repeated treatment by exposing them to a solvent, such as xylene for a period of time. In one aspect of the present invention, the slides are exposed to xylene for five minute durations. This procedure can be repeated several times, such as for three exposures of five minutes each. Then, preferably, the slides are rehydrated by for a duration, such as one minute, to 100%, 95%, 70%, and 50% of an alcohol, such as ethanol, respectively, followed by distilled water. Specimens were then air dried at room temperature.

The FISH procedure utilized the following probe that is specific to the 16S ribosomal RNA sequence of H. spp.: 5′-CAC-ACC-TGA-CTG-ACT-ATC-CCG-3′ conjugated at the 5′ end with FITC (Sigma-Genosis, Inc.). This was dissolved at 5 microgram/ml in Tris-EDTA buffer (10 mM & 1 mM, respectively, pH 7.5), stored at—20° C. and dissolved in 0.9 M NaCl, 0.02 M Tris/HCl, pH 8.0, 0.01% SDS containing 30% formamide at 5 ng/μl as a working stock solution that was used for hybridization to tissue sections. The DNA hybridization reaction occurred by adding 50 μl working stock solution per section and incubation at 46° C. for 90 minutes in a humidified chamber. Following hybridization, the slide was exposed to three successive washes using 0.112 M NaCl, 20 mM Tris/HCl, 0.01% SDS, pH 8.0 and a final wash in 0.1 M phosphate-buffered saline, pH 7 (PBS). Then the specimen was rinsed with distilled water and allowed to air dry.

Three hundred microliters of DAPI (4′, 6-diamidino-2-phenylindole, dilactate; Sigma Chemical Co, catalog number D9564) working dilution (30 nM in PBS) was then placed on top of the sections and incubated at room temperature in the dark. The slide was then washed briefly in distilled water to remove unbound DAPI and the slide was prepared for fluorescent microscopy.

Preferably the fluorescent antibody detection of Helicobacter is performed on successive sections from the same tissue that are spotted and fixed onto glass slides by incubation overnight at 55° C. In one embodiment, these slides are deparaffinized by repeated treatment by exposing them to a solvent, such as xylene for a period of time. In one aspect of the present invention, the slides are exposed to xylene for five minute durations. This procedure can be repeated several times. Then, preferably, the slides are rehydrated by exposure for a duration, such as one minute, to 100%, 95%, 70%, and 50% of an alcohol, such as ethanol, respectively, followed by exposure to distilled water.

In one embodiment, the slides are then soaked in distilled water for a duration, such as five minutes and in PBS for a duration, such as 5 minutes. Preferably, either of these soakings can be repeated consecutively. Non-specific binding is then preferably blocked by incubation for a period, such as thirty minutes, in 20% goat serum (US Biological, catalog number S1003-45) and the excess serum was then decanted. Slides are then preferably incubated in primary antibody, mouse anti-Helicobacter spp. (US Biological, catalog number H1840-11) at 1/100 dilution in PBS for a duration, such as one hour, at room temperature in a humidified chamber. Excess antibody is then preferably washed out by preferably multiple washes with PBS and then the slides are preferably incubated with a secondary antibody, goat-anti-mouse IgG-TRITC conjugate (Jackson Laboratories, Inc; catalog number 115-025-0030) at 1/500 dilution in PBS for a duration, such as one hour, at room temperature in a humidified chamber. This is preferably followed by preferably multiple washes of the slide, such as two, in PBS for a duration, such ten minutes, and then preferably the slides are allowed to dry at room temperature in the dark.

The fluorescent microscopy of stained cells is performed according to known procedures. In one embodiment, 4′-6-Diamidino-2-phenylindole (DAPI) is used to form fluorescent complexes with natural double-stranded DNA, showing a fluorescence specificity for AT, AU and IC clusters. DAPi is a non-specific DNA stain and probably represents nuclear DNA, predominantly. The DAPI binds to the anti-Helicobacter DNA that strongly enhances its fluorescence. In one embodiment, the morphology of the cells nucleus on the slides are examined by fluorescent microscopy using an Olympus Model BX40 instrument and oil emersion at 1000× magnification.

Using different excitation, emission wavelengths, and optical filters, it is possible to distinguish between different fluorescent signals from one specimen or slide. In one embodiment, the excitation wavelength is preferably 360 nm and with an emission wavelength at ≧460 nm. Additionally, the results of FISH for Helicobacter spp. using excitation wavelength at 494 nm and emission wavelength at ≧515 nm that induces fluorescence of the FITC conjugate coupled to the Helicobacter spp.-specific DNA probe. The Helicobacter spp. and DAPI staining show strong coincidence suggesting that all cells within this field are infected with Helicobacter spp. In another embodiment, the fluorescent antibody signal resulting from excitation wavelength at 550 nm and emission wavelength at ≧573 nm which results in excitation of TRITC that is conjugated to the Helicobacter spp. specific monoclonal antibody. The reaction product of the MAB shows a distinct localization that is apparently different from that observed by DAPI staining or the FISH technique, possibly due to reactivity to Helicobacter spp. secreted factors, e.g., virulence factors. Control experiments showed that non-infected tissues were negative for Helicobacter spp. by FISH or FA staining (not shown).

The present invention further provides for methods of diagnosing the association of Helicobacter spp. associated with a number of so called ‘auto-immune’ disease conditions; such as diabetes mellitus, gingivitis, Crohn's disease, and IBD of the canine, cancer, and so on. Physical diagnosis of patients including human, feline, canine, equine and bovine or other animal species by standard methods of medical and veterinary practice. Those patients diagnosed with a non-gastric disease are subject to additional testing to determine the presence of Helicobacter spp. organism or products of the organism or host-derived Helicobacter—specific components within bodily fluids and tissues.

Those patients found to have Helicobacter spp. outside of the lumen of the stomach and leading to pathological processes, ranging from gingivitis to Crohn's disease, diabetes, liver disease, cancer, and so on should be aggressively treated with antibiotics to eliminate the organism in the locations outside of the stomach. A therapy of 90 days has been found to be 95% effective in canines (dogs) with a relapse rate within one year of less than 5%. Useful antibiotics include metronidazole in combination with another antibiotic such as Keflex, tetracycline, clathrimycin, and so on. The small percentage of non-responsive patients are provided with additional therapy. Maintenance with mastic gum (Isle of Chios) may be helpful.

Antibiotic therapy preferably includes treating with metronidazole, Keflex, amoxicillin, tetracycline, clathrimycin, bismuth and the herb, mastic gum or combinations of any of these compounds. The selected antibiotics are administered for a period of preferably between 1 to 120 days. More preferably, the antibiotics are administered for 90 days.

Molecular signal therapy preferably consists of administering molecular components of the Helicobacter spp. as therapeutic agents including for example, killed organism, virulence factors and toxins elaborated by Helicobacter spp. Molecular signaling consists of administering a specific Helicobacter spp. molecular component, vacuolating cytotoxin Vac A, for example, at a preferred dosage of 2 micrograms/cc as I cc administered by subcutaneous injection or by oral drop placed upon the gums or cheek pouch two to six times a day at intervals of at least on hour. The molecular signal therapeutic is prepared at a concentration of 1 to 20 micrograms per ml suspended in bacteriostatic water. Molecular signal therapy may be concurrent with antibiotic therapy (above) without conflicting either therapy. An advantage of the Vac-A and/or CAG is that diseased tissues normalize at the cellular level.

Vaccination consists of evoking an immune response by the patient to a molecular constituent of Helicobacter spp. with appropriate adjuvants if necessary. A preferred method is to evoke an immune response to VacA and/or CAG or other virulence factors since Helicobacter spp. constituents of the GI tract may be maintained while blocking the ability of the Helicobacter spp. to invade other tissues. Since Vac-A derivatives or its derivatives are reported to have immunosuppressant activity thought to suppress calcineurin, the Vac A vaccine of the present invention includes use of Vac-A derivatives that induce neutralization or neutralizing antibody formation while blocking inhibition of calcineurin activity. Vaccination occurs by injection of appropriate concentration of antigen (or antigenic derivative), adjuvant and preservative according to methods known to one skilled in the art and complying with manufacturing standards.

Small interfering RNA (siRNA) and anti-sense therapy has been shown to be an effective method of blocking the expression of specific genes. According to the present invention, siRNA and antisense therapy is targeted to specific Helicobacter spp. sequences that inhibit the invasion or growth within tissues underlying the diseases that have been herein correlated with Helicobacter spp. infection. Such sequences include but are not limited to those regulating the expression of the virulence and toxin factors, vacuolating cytotoxin A, Cag PAI, iceA, babA and other unidentified factors.

Receptor-based therapy includes interventions that inhibit interactions between Helicobacter spp. virulence factors and toxins that preclude the invasion of Helicobacter spp. into host tissues. Receptors include protein tyrosine phosphatase receptor type Z that have been shown to mediate interaction between Vac A and host cells thereby playing a critical role in the invasion of Helicobacter spp. into tissues. (Nature Genetics, 02/03 from BioWorld Feb. 27, 2003)

Therapy according to the present invention includes stand-alone Helicobacter spp. eradication measures, combined Helicobacter therapies or combined therapy including—various different Helicobacter spp. eradication therapies together (or alone) with standard therapies of carcinoma, sarcoma, mast cell cancer, lymphoma, melanoma, Epuli, diabetes mellitus, gingivitis and periodontitis, peptic and duodenal ulcers or colonization, chronic bowel disease, Crohn's disease, spinal demyelinization, multiple sclerosis, Lupus like diseases, degenerative kidney disease, benign prostatic hypertrophy, cardiomyopathy, macular degeneration, retinitis, glaucoma, rheumatoid arthritis degenerative liver disease, diabetes mellitus (Type I diabetes), periodontal disease and gingivitis, gastroesophageal reflux disease (GERD), and various other immune-mediated diseases.

### EXAMPLE 1

### Detection of Helicobacter Organism within Tissues

Tissues were extracted from a 10-year old canine patient exhibiting symptoms of chronic gingivitis including plasmacytic and lymphocytic invasion, discoloration and retraction of gums, etc. The tissue was fixed in 10% formalin and 2-micron thick sections were prepared from paraffin-embedded material by standard methods.

Tissue sections were then spotted and fixed onto glass slides by incubation overnight at 55° C., deparaffinized by xylene treatment (three exposures of 5-minutes each) and rehydrated by 1-minute exposure to 100%, 95%, 70%, and 50% ethanol, respectively, followed by distilled water. Slides were then air dried at room temperature.

The FISH procedure utilized the following fluorescent probe that is specific to the 16S ribosomal RNA sequence of Helicobacter spp.: 5′-CAC-ACC-TGA-CTG-ACT-ATC-CCG-3″ conjugated at the 5′ end with FITC (Sigma-Genosis, Inc.). This was dissolved at 5 microgram/ml in Tris-EDTA buffer (10 mM & 1 mM, respectively, pH 7.5), stored at −20° C. and dissolved in 0.9 M NaCl, 0.02 M Tris/HCl, pH 8.0, 0.01% SDS containing 30% formamide at 5 ng/μl as a working stock solution that was used for hybridization to tissue sections. The DNA hybridization reaction occurred by adding 50 μl working stock solution per section and incubation at 46° C. for 90 minutes in a humidified chamber. Following hybridization, the slide was exposed to three successive washes using 0.112 M NaCl, 20 mM Tris/HCl, 0.01% SDS, pH 8.0 and a final wash in 0.1 M phosphate-buffered saline, pH 7 (PBS). Then the slide was rinsed with distilled water and allowed to air dry. This procedure will determine whether there exists unspecified Helicobacter spp. DNA in a patient's tissue specimen.

Three hundred microliters of DAPI (4′, 6-diamidino-2-phenylindole, dilactate; Sigma Chemical Co, catalog number D9564) working dilution (30 nM in PBS) was then placed on top of the sections and incubated at room temperature in the dark. The slide was then washed briefly in distilled water to remove unbound DAPI and the slide was prepared for fluorescent microscopy. This procedure will identify those areas of the patient's tissue that is infected with specific Helicobacter spp. DNA.

Fluorescent antibody detection of Helicobacter was performed on successive sections from the same tissue that were spotted and fixed onto glass slides by incubation overnight at 55° C., deparaffinized by xylene treatment (three exposures of 5-minutes each) and rehydrated by 1-minute exposure to 100%, 95%, 70%, and 50% ethanol, respectively, followed by distilled water. Slides were soaked twice in distilled water (5 minutes) and twice in PBS (5 minutes). Non-specific binding was then blocked by 30-minute incubation in 20% goat serum (US Biological, catalog number S1003-45) and excess serum was then decanted. Slides were then incubated in primary antibody, mouse anti-Helicobacter spp. (US Biological, catalog number H1840-11) at 1/100 dilution in PBS for one hour at room temperature in a humidified chamber. Excess antibody was then washed out by two washes with PBS and the slide was incubated with secondary antibody, goat-anti-mouse IgG-TRITC conjugate (Jackson Laboratories, Inc; catalog number 115-025-0030) at 1/500 dilution in PBS for one hour at room temperature in a humidified chamber. This was followed by two washes of 10 minutes each in PBS and the slide was then allowed to dry at room temperature in the dark. This procedure specifically recognizes the Helicobacter spp. antibody in a patient's tissue specimen.

The slides were examined by fluorescent microscopy using an Olympus Model BX40 instrument; oil emersion at 1000× magnification.

### EXAMPLE 2

### Treatment of Helicobacter Infection with Antibiotics

Antibiotic therapy consists of a 90-day course of ½ to full-dose metronidazole plus full-dose broad-spectrum antibiotic including, but not limited to, Keflex, amoxicillin, tetracycline, clathrimycin, bismuth and the herb, mastic gum. This therapy has been found to be effective in treating Helicobacter spp. infections outside or involving the upper GI tract. A 90-day course of therapy is preferable for optimal success and preventing recurrence. This treatment regimen has been found to be 95% effective with a relapse rate less than 5% after 12-24 months in the treatment of over 130 canine and feline patients.

### EXAMPLE 3

### Treatment of Helicobacter Infection with Antibiotics

Helicobacter spp. organisms have been identified by FA tissue treatment and examination in canine, feline and human cases of gingivitis and periodontitis, in canine chronic (Crohn's) disease intestinal tissues, the pancreatic tissues of feline, canine and human diabetes mellitus tissues, the cardiac tissue of humans with cardiomyopathy, and a broad spectrum of canine, feline and human malignant cancer tissues, primary and metastatic. Positive detection in the canine was based upon screening of circulating antibodies to Helicobacter spp. Canine and feline cases were then treated with the following antibiotic regime: ½ dose metronidazole or fenbendazole plus therapeutic dosages of Keflex, amoxicillin, or tetracycline that were administered for 90 days.

In canine patients with gingivitis, receding gums, and periodontitis, the following were observed in response to antibiotic therapy: classical foul halitosis disappeared with 48-72 hours of the beginning of antibiotic therapy. The treatment of approximately 130 canine cases of oral Helicobacter spp. infection in the manner described and following routine dental prophylaxis resulted in a tightening of tooth root attachment, cessation of gum contraction and further root exposure and healing of the gingival tissues except in cases where concomitant autoimmune complex exists. Also, there was a marked reduction in the rates of accumulation of tartar without the use of dental abrasives, flossing or brushing.

Antibiotic treatment of approximately 130 cases of gingivitis and periodontitis for 90 days resulted in a 95% re-establishment of solid root attachment, moderate gum growth covering exposed roots and amelioration of gingivitis with a relapse rate of approximately 5% after 12 months.

The example of 18 cases of chronic bowel disease (Crohn's) or inflammatory bowel disease (IBD) in the canine, with chronic diarrhea that was sometimes hemorrhagic, demonstrated restoration of normal fecal formation without diet changes following a 90-day course of antibiotics as described above. About 10% of these cases failed to exhibit solid bowel movements due to damage from scarring of the bowel tissues.

The example of 31 cases of duodenitis-gastritis syndrome in canines with NSAID (aspirin) sensitivity and reflux disease associated with Helicobacter spp. were corrected 90% of the time by antibiotic therapy for 90 days as described. The remaining 10% of the chronic cases had sufficient damage to the esophagus that full recovery from GERD was not noted, however, it was much improved and easily controlled by use of antacid compounds. These same patients also demonstrated a consistent, compulsive desire to consume grass with resulting emesis, in many cases. These patients benefited when given antacids, mastic gum, and/or bismuth-containing oral medications and compounds.

The association of Helicobacter spp. with a broad spectrum of so-called “auto-immune” conditions is extensive. The association of Helicobacter spp. with these conditions was determined by use of fluorescent antibody to Helicobacter spp. as described previously (Example 1). These conditions include: pemphigoid diseases, demyelinization of the spinal cord, atopy, macular degeneration, retinitis, glaucoma, chronic pulmonary disease, degenerative kidney disease, and chronic liver diseases. The association of Helicobacter spp. with malignant cancer has been consistent in all human, feline, and canine tissues studied, and include carcinoma, sarcoma, mast cell cancer, lymphoma, melanoma, and epuli.

Of the 179 cases sited above, most of which are canine, approximately 5% experienced relapses within 12-24 months after the 90-day course of antibiotics. This relapse rate was further reduced and benefited with the therapeutic Vac A injection/oral drop regimen was instituted and maintained (See example 3).

### EXAMPLE 4

### Treatment of Helicobacter Infection with Vac A

Vacuolating Toxin A (recombinant Vac A; Austral Biologicals, catalog number HPA-5010-4) at 0.01 mg/ml in bacteriostatic water and 0.2 ml was injected subcutaneously 2-6 times per day at intervals of at least one hour to patients with various conditions associated with evidence of Helicobacter infection.

Seventy percent of 28 patients with pemphygus, allergic dermatitis and atopy were either corrected or markedly improved when Vac A was used as sole therapy. In some more chronic and persistent cases, diphenhydramine (25 to 75 mg bid/size of patient) and other anti-histamines was used successfully to supplement the Vac A therapy.

Eighty percent of over 80 predominantly canine patients with cardiomyopathy, chronic pulmonary disease, malignant cancers, demyelinization (MS), diabetes mellitus, degenerative kidney and liver disease demonstrated reversal (15%), arrest (30%) or slowing (30%) of progressive disease processes when treated with Vac A as described above.

Although there has been described what is at present considered to be the preferred embodiments of methods of diagnosis, treatment, and vaccination of Helicobacter spp. associated infections, it will be understood that these methods can be embodied in other specific forms without departing from the spirit or essential characteristics thereof. For example, additional or different solutions, other than those described herein, for detecting the Helicobacter spp. may be used. Also, other light source excitation and emission wavelengths may be used other than those described herein without departing from the inventive novelty described herein. The present embodiments are, therefore, to be considered in all aspects as illustrative and not restrictive. The scope of the invention is indicated by the appended claims rather than the foregoing description.

What is claimed:

1. A method for determining the cause of a diagnosed non-gastric disease in a patient, comprising:
obtaining a tissue specimen related to said diagnosed non-gastric disease from said patient;
synthesizing a single strand of a double strand DNA Helicobacter specific nucleotidic sequence of said tissue specimen for binding a fluorescent moiety on said single strand of said double strand of DNA Helicobacter specific nucleotidic sequence;
denaturing said DNA Helicobacter specific nucleotidic sequence to separate said double strand of DNA to allow access of said fluorescent moiety to their respective nucleotidic sequence;
detecting said tissue specimen for the presence of a Helicobacter spp.; and
associating a presence of said Helicobacter spp. with said diagnosed non-gastric disease selected from the group consisting of carcinoma, sarcoma, mast cell cancer, lymphoma, melanoma, Epuli, diabetes mellitus, gingivitis and periodontitis, peptic and duodenal ulcers or colonization, chronic bowel disease, Crohn's disease, spinal demyelinization and/or multiple sclerosis, Lupus like diseases, degenerative kidney disease, benign prostatic hypertrophy, cardiomyopathy, macular degeneration, retinitis, glaucoma, rheumatoid arthritis, degenerative liver disease, diabetes mellitus (Type I diabetes), periodontal disease and gingivitis, gastroesophageal reflux disease (GERD), and various other immune-mediated diseases.

2. The method for determining the cause of a diagnosed non-gastric disease of claim 1 wherein said fluorescent moiety is selected from the group consisting of FITC, 6FAM, TRITC, Texas red, and rhodamine.

3. The method for determining the cause of a diagnosed non-gastric disease of claim 1 wherein said binding a fluorescent moiety comprises:
covalently attaching said fluorescent moiety to the 5′ end of the DNA sequence 5′-CAC-ACC-TGA-CTG-ACT-ATC-CCG-3′ of said nucleotidic sequence.

4. The method for determining the cause of a diagnosed non-gastric disease of claim 1 wherein said binding a fluorescent moiety comprises:
covalently attaching said fluorescent moiety to the 5′ end of the DNA sequence 5′-GCC-GTG-CAG-CAC-CTG-TTT-TCA-3′ of said nucleotidic sequence.

5. The method for determining the cause of a diagnosed non-gastric disease of claim 1 wherein said detecting said tissue specimen comprises:
subjecting said fluorescent moiety of said tissue specimen to a light source having an excitation wavelength to cause said fluorescent moiety emit an emission wavelength.

6. The method for determining the cause of a diagnosed non-gastric disease of claim 5 wherein said excitation wavelength is substantially between 280 nm and 650 nm.

7. The method for determining the cause of a diagnosed non-gastric disease of claim 5 wherein said detecting said tissue specimen further comprises:
detecting said emission wavelength to determine the presence of Helicobacter spp.

8. The method for determining the cause of a diagnosed non-gastric disease of claim 1 wherein said nucleotidic sequences is selected from the group consisting of Helicobacter species-specific sequences, Helicobacter genus-specific sequences, and sequences binding to antibiotic resistant strains of Helicobacter spp.

9. The method for determining the cause of a diagnosed non-gastric disease of claim 1 further comprising:
detecting, using a fluorescent antibody diagnostic method, the presence of antigens of protein constituents of Helicobacter spp. associated with said tissue specimen.

10. The method for determining the cause of a diagnosed non-gastric disease of claim 9 further comprising:
detecting, using a fluorescent antibody diagnostic method, the presence of virulence factors and toxins protein factors elaborated by said Helicobacter spp.

11. The method for determining the cause of a diagnosed non-gastric disease of claim 10 wherein said virulence factors are selected from the group consisting of vaculating cytotoxin A, Cag, PAI, iceA, and babA.

12. The method for determining the cause of a diagnosed non-gastric disease of claim 1 wherein said tissue specimen further includes bodily fluids of said patient.

13. The method for determining the cause of a diagnosed non-gastric disease of claim 1 wherein said tissue specimen further includes tumor cells of said patient.

14. The method for determining the cause of a diagnosed non-gastric disease of claim 1 wherein said Helicobacter spp. is selected from the group consisting of Helicobacter pylori, Helicobacter heilmannii, Helicobacter fells, Helicobacter mustelae, Helicobacter bizzozeronii, Helicobacter salomonis, Helicobacter canis, Helicobacter cinaedi, Helicobacter fexispira, Helicobacter fennelliae, Helicobacter nemestrinae, Helicobacter dinaedii, Helicobacter pametensis, Helicobacter bilis, and F. R. Rappin.

15. A method for treating the cause of a diagnosed non-gastric disease in a patient, comprising:
detecting the presence of a Helicobacter spp. associated with said diagnosed non-gastric disease in at least one of bodily tissues and fluids of said patient; and
administering a treatment for a duration to said patient.

16. The method for treating the cause of a diagnosed non-gastric disease of claim 15 wherein said treatment is selected from the group consisting of antibiotic therapy, molecular signal therapy, vaccination, interference with receptor systems mediating Helicobacter spp. invasion into tissues, and use of small-interfering RNA sequences to specifically block virulence or replication of Helicobacter spp.

17. The method for treating the cause of a diagnosed non-gastric disease of claim 15 wherein said duration is between 60 days and 120 days.

18. The method for treating the cause of a diagnosed non-gastric disease of claim 15 wherein said duration is 90 days.

19. The method for treating the cause of a diagnosed non-gastric disease of claim 16 wherein said antibiotic therapy comprises:
administering to said patient an at least one antibiotic selected from the group consisting of metronidazole, Keflex, amoxicillin, tetracycline, clathrimycin, bismuth and the herb, mastic gum or combinations of any of these compounds to said patient.

20. The method for treating the cause of a diagnosed non-gastric disease of claim 16 wherein said molecular signal therapy comprises:
administering to said patient a molecular component of said Helicobacter spp.

21. The method for treating the cause of a diagnosed non-gastric disease of claim 20 wherein said molecular component is selected from the group consisting of killed organism, virulence factors, and toxins elaborated by said Helicobacter spp.

22. The method for treating the cause of a diagnosed non-gastric disease of claim 15 wherein said vaccination comprises:
administering to said patient a molecular constituent of Helicobacter spp. to evoke an immune response by said patient.

23. The method for treating the cause of a diagnosed non-gastric disease of claim 16 wherein said use of small-interfering RNA sequences to specifically block virulence or replication of Helicobacter spp. comprises:
administering to said patient said small-interfering RNA sequences to regulate the expression of the virulence and toxin factors of said Helicobacter spp.

24. The method for treating the cause of a diagnosed non-gastric disease of claim 16 wherein said virulence and toxin factors include vacuolating cytotoxin A, Cag PAI, iceA, and babA.

25. The method for treating the cause of a diagnosed non-gastric disease of claim 15 wherein said detecting the presence of said Helicobacter spp. comprises:
analyzing said at least one bodily tissue and fluids of said patent by fluorescent in-situ hybridization analysis.

26. The method for treating the cause of a diagnosed non-gastric disease of claim 15 wherein said diagnosed non-gastric disease is selected from the group consisting of carcinoma, sarcoma, mast cell cancer, lymphoma, melanoma, Epuli, diabetes mellitus, gingivitis and periodontitis, peptic and duodenal ulcers or colonization, chronic bowel disease, Crohn's disease, spinal demyelinization and/or multiple sclerosis, Lupus like diseases, degenerative kidney disease, benign prostatic hypertrophy, cardiomyopathy, macular degeneration, retinitis, glaucoma, rheumatoid arthritis, degenerative liver disease, diabetes mellitus (Type I diabetes), periodontal disease and gingivitis, gastroesophageal reflux disease (GERD), and various other immune-mediated diseases.

27. A test kit for analyzing a tissue specimen of a patient for the presence of Helicobacter spp. DNA related to a diagnosed non-gastric disease of said patient, comprising:
a nucleotidic sequence of said Helicobacter spp. DNA for binding a fluorescent moiety on said single strand;
an agent for hybridizing said DNA nucleotidic sequence for annealing said hybridizing agent to said nucleotidic sequence; and
an agent for washing said DNA nucleotidic sequence to remove any unannealed hybridizing agent from said nucleotidic sequence.

28. The test kit for analyzing a tissue specimen of a patient of claim 27 further comprising:
an agent to deparaffinize said tissue specimen that is stored in at least one of formalin and paraffin.

29. The test kit for analyzing a tissue specimen of a patient of claim 27 wherein said tissue specimen is tumor cells from said patient.
